(19) United States
(12) Patent Application Publication
(10) Pub. No.: US 2023/0172513 A1
(43) Pub. Date: Jun. 8, 2023

(54) FLEXIBLE, INSERTABLE, TRANSPARENT MICROELECTRODE ARRAY FOR DETECTING INTERACTIONS BETWEEN DIFFERENT BRAIN REGIONS

(71) Applicant: The Regents of the University of California, Oakland, CA (US)

(72) Inventors: Duygu Kuzum, La Jolla, CA (US); Yichen Lu, Richmond, CA (US); Xin Liu, San Diego, CA (US); Jeong-hoon Kim, La Jolla, CA (US)

(21) Appl. No.: 18/063,040

(22) Filed: Dec. 7, 2022

Related U.S. Application Data
(60) Provisional application No. 63/287,015, filed on Dec. 7, 2021.

(51) Int. Cl. A61B 5/293; B81B 1/00; B81C 1/00; A61B 5/00

(52) U.S. Cl. CPC A61B 5/293; A61B 5/0084; B81B 1/008; B81C 1/00111; A61B 2562/04; A61B 2562/028; A61B 2562/125; A61B 2562/164; A61B 2562/0209; B81B 2201/12; B81B 2203/04; B81B 2203/0361

(57) ABSTRACT

Flexible, insertable, transparent microelectrode arrays that allow integration of electrophysiological recordings with any optical imaging or stimulation technology are disclosed. In some embodiments of the disclosed technology, a microelectrode array includes a flexible substrate layer including a shank member extending in a first direction and a tapered tip at an end of the shank member, and a plurality of electrode wires arranged in the first direction on the flexible substrate layer, wherein the plurality of electrode wires includes adjacent electrode wires having different lengths from each other such that an electrode wire arranged closer to a centerline of the flexible substrate layer is longer than an adjacent electrode arranged further away from the centerline of the flexible substrate.

### PRIORITY CLAIM AND CROSS-REFERENCE TO RELATED APPLICATIONS

This patent document claims the priority and benefits of U.S. Provisional Application No. 63/287,015, titled “FLEXIBLE, INSERTABLE, TRANSPARENT MICROELECTRODE ARRAY FOR DETECTING INTERACTIONS BETWEEN DIFFERENT BRAIN REGIONS” filed on Dec. 7, 2021. The entire content of the aforementioned patent application is incorporated by reference as part of the disclosure of this patent document.

### STATEMENT REGARDING FEDERALLY SPONSORED RESEARCH OR DEVELOPMENT

This invention was made with government support under N00014161253 awarded by the Navy Office of Naval Research, EB026180 awarded by the National Institutes of Health, and ECCS2024776 awarded by the National Science Foundation. The government has certain rights in the invention.

### TECHNICAL FIELD

The technology and implementations disclosed in this patent document generally relate to devices that can detect neural activity.

### BACKGROUND

Brain computations often require interactions between different cortical and subcortical structures. Understanding of these long-range interactions in the brain requires monitoring of simultaneous activity patterns across these areas. This couldbe achieved by simultaneous multimodal recordings combining electrophysiological recordings and large-scale functional optical imaging. However, seamless integration of optical imaging with electrophysiology is difficult with conventional microelectrodes because large probe shanks made of rigid and opaque materials can prevent lowering of the microscope objective and block the field of view of imaging.

### SUMMARY

The disclosed technology can be implemented in some embodiments to provide a flexible, insertable, transparent microelectrode array that allows integration of electrophysiological recordings with any optical imaging or stimulation technology.

In some implementations of the disclosed technology, a microelectrode array includes a flexible substrate layer including a shank member extending in a first direction and a tapered tip at an end of the shank member; and a plurality of electrode wires arranged in the first direction on the flexible substrate layer, wherein the plurality of electrode wires includes adjacent electrode wires having different lengths from each other such that an electrode wire arranged closer to a centerline of the flexible substrate layer is longer than an adjacent electrode arranged further away from the centerline of the flexible substrate.

In some implementations of the disclosed technology, a method of fabricating a microelectrode array includes forming a substrate layer that includes a shank member extending in a first direction and a tapered tip at an end of the shank member; transferring a transparent electrode layer formed on a base substrate onto the substrate layer; and forming a plurality of electrode wires arranged in the first direction on the substrate layer by at least patterning the transparent electrode layer, wherein the plurality of electrode wires includes adjacent electrode wires having different lengths from each other such that an electrode wire arranged closer to a centerline of the substrate layer is longer than another electrode wire that is arranged further away from the centerline of the substrate layer.

In some implementations of the disclosed technology, a microelectrode array includes a flexible substrate layer extending in a first direction and including a tapered tip at an end of the flexible substrate layer; a plurality of electrode wires arranged in the first direction at an interval on the flexible substrate layer, wherein the plurality of electrode wires includes a first electrode wire arranged along a centerline of the flexible substrate layer and a second electrode wire arranged along an edge of the flexible substrate layer, wherein the first electrode wire is longer than the second electrode wire; and an encapsulation layer disposed over the plurality of electrode wires and including one or more electrode openings structured to expose a portion of one or more electrode wires.

In some implementations of the disclosed technology, a microelectrode array includes a substrate layer including a flexible tapered shank member structured to include a recording tip at an end of the flexible tapered shank member, and a plurality of electrodes arranged on the substrate layer, wherein the electrodes arranged on the shank member of the substrate layer are spaced apart from each other at an interval and have different lengths such that an electrode wire arranged along a centerline of the shank member is longer than another electrode wire arranged along an edge of the shank member. In some implementations of the disclosed technology, the microelectrode array further includes an encapsulation layer disposed over the electrodes, wherein the encapsulation layer includes one or more electrode openings structured to expose a portion of each electrode.

The above and other aspects and implementations of the disclosed technology are described in more detail in the drawings, the description and the claims.

### BRIEF DESCRIPTION OF THE DRAWINGS

FIGS. 1A-1N show an example of flexible, insertable, transparent microelectrode array.

FIGS. 2A-2E show simultaneous multimodal recordings from the hippocampus and cortex.

FIGS. 3A-3B show the neuron spike waveforms in different recording sessions from one mouse.

FIGS. 4A-4G show signal-to-noise ratio (SNR) for the spikes, LFPs and wide-field fluorescence.

FIGS. 5A-5E show cortical activity onset tends to precede sharp-wave ripples (SWRs).

FIGS. 6A-6E show diverse SWR-associated cortical activity patterns.

FIGS. 7A-7E show different cortical activity patterns associated with distinct hippocampal neuronal activity patterns during SWRs.

FIGS. 8A-8D show microscope pictures of different flexible, insertable, transparent microelectrode array probe designs.

FIGS. 9A-9C show testing the multimodal recording setup using flexible, insertable, transparent microelectrode array and standard silicon probes under both the wide-field and 2-photon imaging systems.

FIGS. 10A-10C show implantation of flexible, insertable, transparent microelectrode array to hippocampus in in vivo experiments and the spike waveforms of example neurons.

FIGS. 11A-11B show SWR-associatedlarge-scale cortical activity.

FIG. 12 shows the distribution of time differences between SWR onset and activity onset in each cortical region.

FIGS. 13A-13F show two-stage TCA algorithm.

FIGS. 14A-14B show the two-stage TCA result and the cortical activation timing analysis for two patterns.

FIG. 15 shows the decoding accuracy of all cortical pattern pairs in each animal.

FIGS. 16A-16B show discriminant neurons in decoding cortical pattern identity and the fraction of distinguishable pairs using different neuron populations.

FIGS. 17A-17D show different cortical activity patterns associated with distinct hippocampal neuronal activity patterns during all SWRs.

FIG. 18 shows simultaneous multimodal wide-field calcium imaging and surface potential recordings.

FIG. 19 shows schematic of the decoding model.

FIG. 20 shows decoding the activities of multiple cortical regions.

FIG. 21 shows decoding of the pixel-level cortex-wide brain activity.

FIG. 22 shows an example method of fabricating a microelectrode array based on some embodiments of the disclosed technology.

### DETAILED DESCRIPTION

The disclosed technology can be implemented in some embodiments to provide an implantable brain electrode that allows recording interactions between different cortex regions or interactions of cortex with other subcortical structures. The disclosed technology can be implemented in some embodiments to provide a flexible, insertable, transparent microelectrode array (FITM array, Neuro-FITM) that allows integration of electrophysiological recordings with any optical imaging, such as high resolution multiphoton imaging, or stimulation technology, such as optogenetics.

In some embodiment, FITM array can be implanted into deep cortical layers and subcortical structures. The flexible probe shank of FITM array can be bent to the side to allow lowering of the microscope objective. Optical transparency of the shank provides a clear field of view and prevents optical shadows or additional noise in optical signals. Low impedance of FITM array provides reliable recordings of local field potentials (LFPs), high-frequency oscillations and single units with a high signal-to-noise ratio (SNR).

In some embodiment, FITM array has an optical transparency, which is important for seamless integration of electrophysiological recordings and optical imaging in multimodal experiments. That integration allows recording brain activity across very large areas in multiple spatial and temporal scales.

In some embodiment, the high flexibility of FITM array allows bending of the probe shank away to lower the microscope objective for two-photon imaging, whereas the rigid shanks of neural electrodes in some implementations prevent lowering of the microscope objective to its working distance. Wide-field microscope images show that the neural electrodes in some implementations block the field of view and generate shadows.

In some embodiment, vertical implantation of FITM array is critical for not blocking the light pathway during optical imaging and minimizing implantation damage. To implant Neuro-FITM arrays vertically without using a rigid shuttle or adding a bioresorbable stiffening layer, the disclosed technology can be implemented in some embodiments to determine the geometry and length of the microelectrode array by performing mechanical analysis to prevent buckling during insertion. Furthermore, the probe can be designed to include additional micromanipulator pads to maximize insertion force against buckling.

Some embodiments of the disclosed can be used to study interactions between the hippocampus and entire cortical regions, and hippocampus communicates with different cortical regions in a selective and diverse manner during a brain rhythm (SWRs) which is crucial for learning and memory.

In some embodiments of the disclosed technology, a microelectrode array includes a substrate layer including a flexible tapered shank member structured to include a recording tip at an end of the flexible tapered shank member, and a plurality of electrodes arranged on the substrate layer, wherein the electrodes arranged on the shank member of the substrate layer are spaced apart from each other at an interval and have different lengths such that an electrode wire arranged along a centerline of the shank member is longer than another electrode wire arranged along an edge of the shank member.

In some implementations, the microelectrode array further includes an encapsulation layer disposed over the electrodes, wherein the encapsulation layer includes one or more electrode openings structured to expose a portion of each electrode. In some implementations, the exposed portion of each electrode includes an end of each electrode arranged at the end of the shank member or along the edge of the shank member.

In some implementations, a substrate layer may include a plurality of shank members, each of which includes a tapered tip at an end thereof. In one example, the shank members extend in the same direction. In another example, some of the shank members extend in a direction different from other shank members. In some implementations, a substrate layer may include 4-8 shank members. In one example, each shank may have 64-128 electrodes, and thus the total number of electrodes may be 512-1024 electrodes.

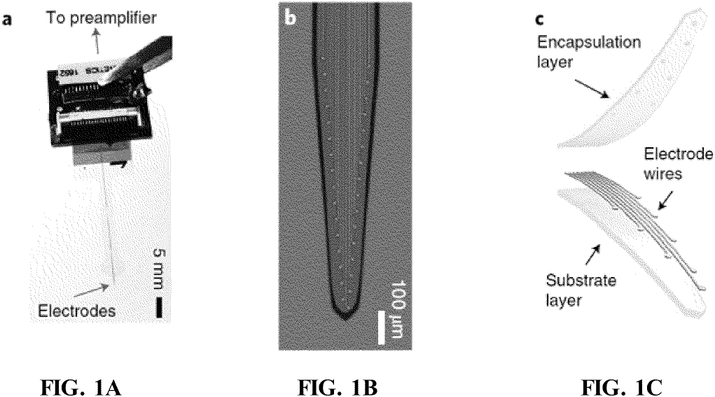

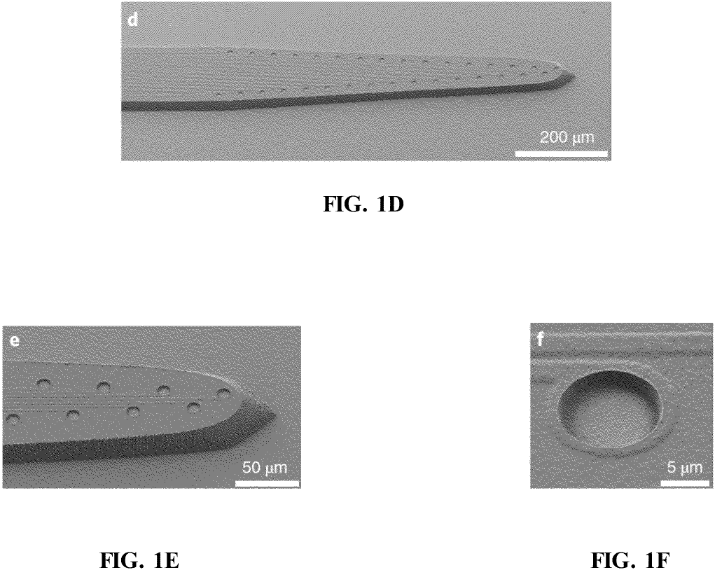

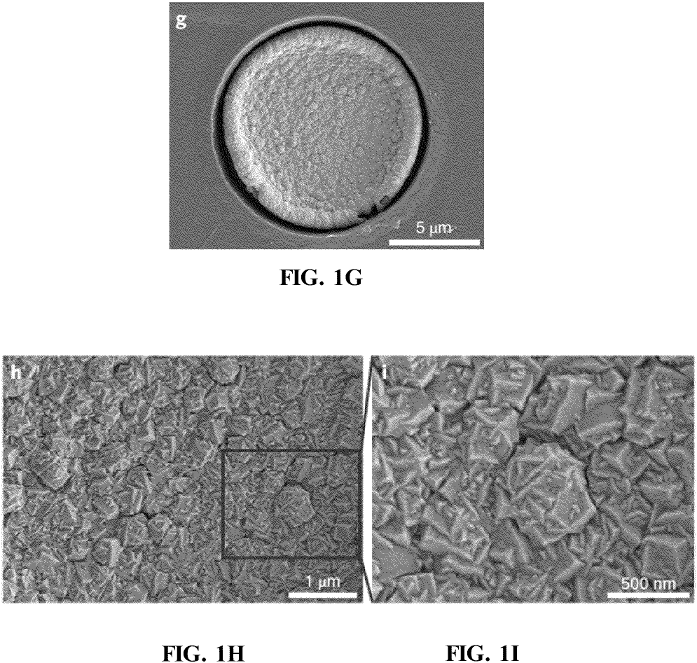

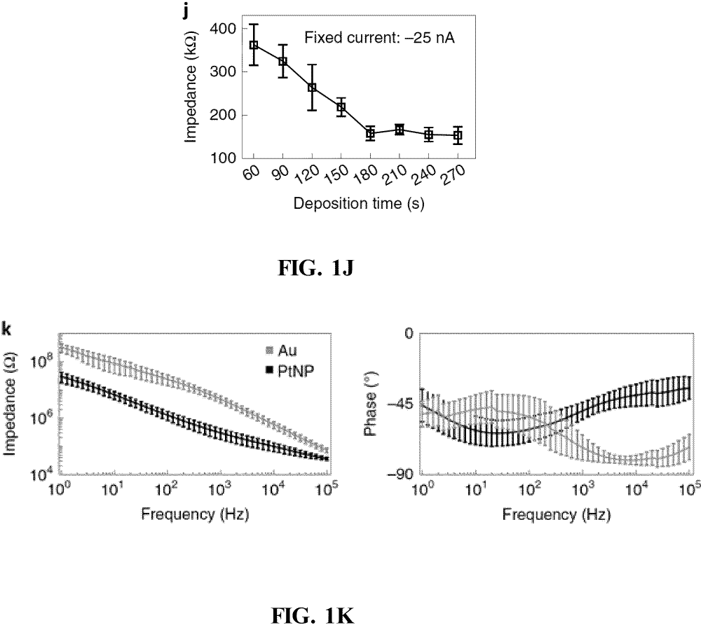

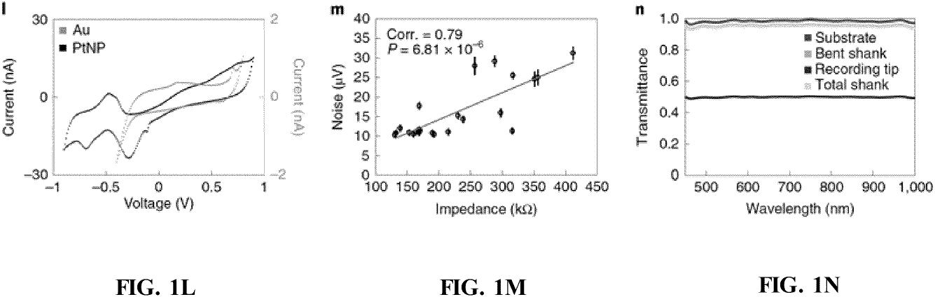

FIGS. 1A-1N show an example of flexible, insertable, transparent microelectrode array (FITM array). FIG. 1A shows an example of FITM array connected to the customized printed circuit board. FIG. 1B is a microscope image showing the layout of the microelectrode array. FIG. 1C is a schematic showing exploded view of the three-layered structure of FITM array. FIGS. 1D-1F are SEM images of the array showing 10-µm-diameter microelectrode openings and 2-µm-wide wires connecting to the microelectrodes. FIG. 1D is an array tip showing the arrangement of microelectrodes. FIG. 1E shows 10-µm-diameter microelectrodes and 2-µm-wide wires encapsulated with 2-µm-thick Parylene-C. FIG. 1F is a magnified view of a single microelectrode and its connected wires encapsulated with 2-µm-thick Parylene-C. FIGS. 1G-1I are SEM images showing PtNPs deposited on to the Au microelectrodes. FIG. 1G shows PtNPs deposited on Au microelectrode. FIG. 1H shows surface and grains of PtNPs and FIG. 1I shows a magnified view of FIG. 1H. FIG. 1J shows electrode impedance as a function of deposition time duringPtNP deposition (mean ± s.d., n = 3 electrodes for a deposition time of 60, 90, 180, 210 and 270 s; n = 4 electrodes for 120, 150 and 240 s of deposition time). FIG. 1K shows EIS magnitude (left) and phase (right) compared between Au and PtNP-deposited Au electrodes. PtNPs reduce the impedance of Au electrodes. The phase plot shows that PtNP electrodes are more resistive at higher frequency ranges than Au electrodes, consistent with the reduction in the impedance magnitude (mean ± s.d., n = 26 electrodes for Au and n = 21 electrodes for PtNP). FIG. 1L shows cyclic voltammetry characteristics ofPtNP-deposited electrodes showing redox peaks corresponding to electrochemical reactions of Pt, indicating an active engagement of PtNPs in the redox processes at the electrochemical interface. FIG. 1M shows noise level for electrodes with different impedances measured in 0.1 M phosphate-buffered saline solution. Recorded signals are first high-pass filtered at 5 Hz and chunked into nonoverlapping 1-s segments. The noise level for each segment is defined as its root mean square value. Each dot marks the mean noise level for each recording channel. The error bar marks the standard error of the mean (s.e.m.) for n = 87 measurements. The noise levels are higher for electrodes with higher impedance (two-sided Student’s t-test, P = 6.81 × 10⁻⁶, n = 23, degree of freedom = 21, “Corr.” indicates correlation). FIG. 1N shows transmittance of the substrate, the bent shank, the recording tip and the total shank as a function of wavelength.

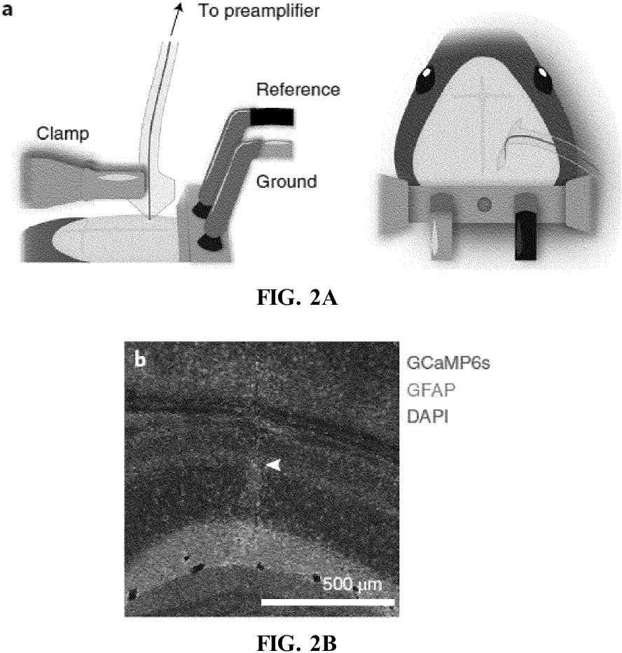

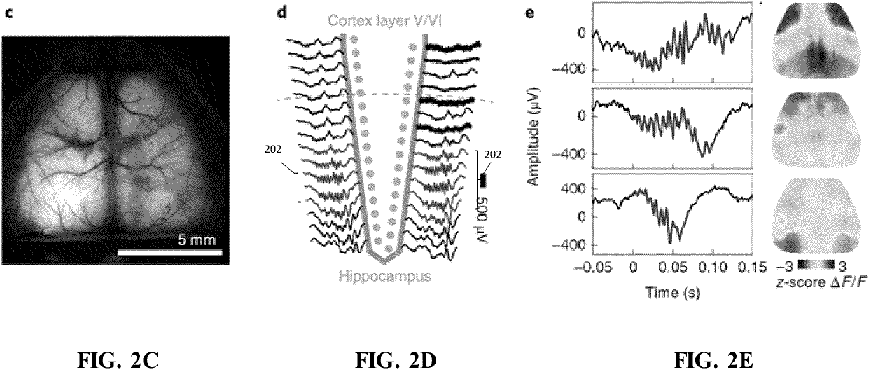

FIGS. 2A-2E show simultaneous multimodal recordings from the hippocampus and cortex. FIG. 2A shows surgical setup. An example of FITM array (Neuro-FITM) is first inserted into the hippocampus (left) and then the shank is bent down to the right side to allow lowering of the microscope objective and clearing of the field of view for imaging (right). FIG. 2B shows penetrating trajectory of Neuro-FITM in the hippocampus visualized by immunostaining against GFAP. The arrowhead indicates the trajectory in the CA1 pyramidal layer. FIG. 2C shows field of view of wide-field calcium imaging during the experiment. Note that the array shank is largely invisible and generated minimal shadows on the overlaying cortex. FIG. 2D shows representative LFP recordings from the channels of the Neuro-FITM probe in one recording session. Multiple channels adjacent (202) to the pyramidal layer of CA1 detected SWRs. FIG. 2E shows examples of simultaneously recorded hippocampal SWRs (left column) and cortical activity (right column, single image frames at SWR onset). Cortical activity shows diverse spatial patterns during SWRs.

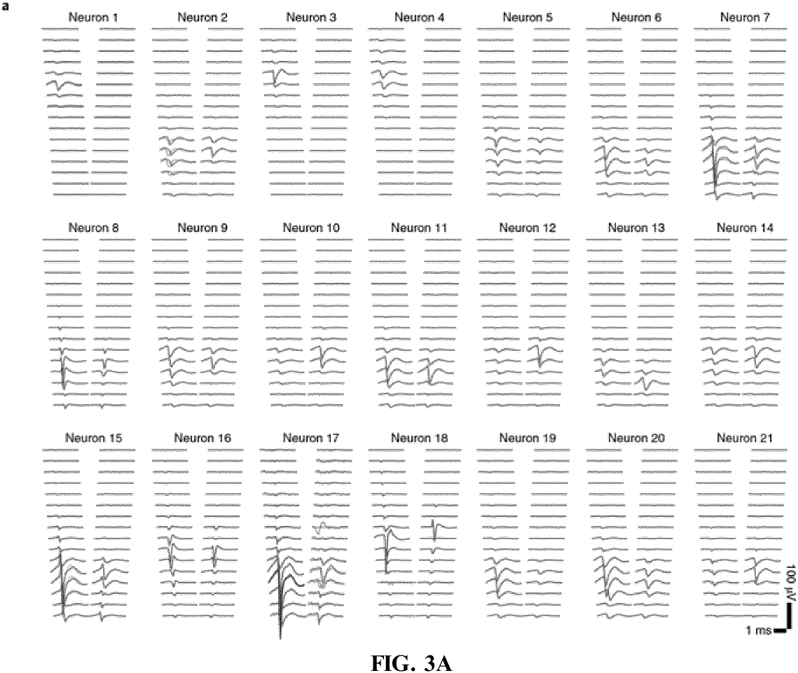

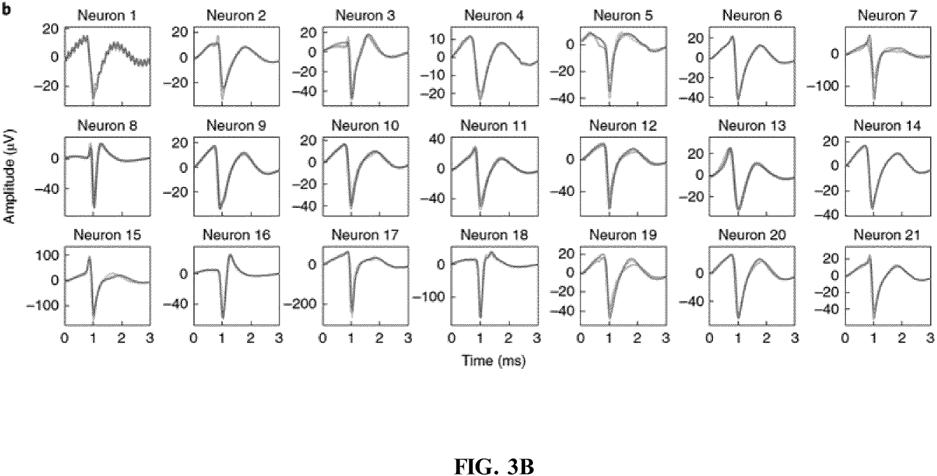

FIGS. 3A-3B show the neuron spike waveforms in different recording sessions from one mouse. FIG. 3A shows spatial profiles of spike waveforms of all 21 neurons recorded across 32 channels in three recording sessions marked by three different colors. Many neurons exhibit stable waveforms that are most prominent in adjacent channels. FIG. 3B shows spike waveforms of all 21 neurons from the channel with the largest amplitude recorded in three sessions. Different colors indicate different recording sessions, as in FIG. 3A. The waveforms of the same neuron recorded at different sessions are highly similar.

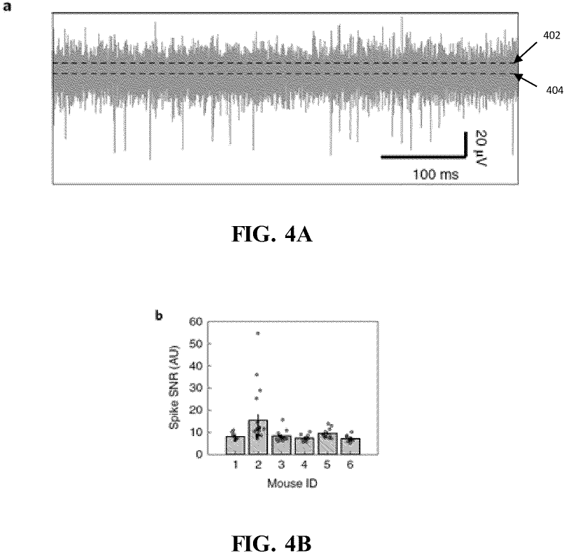

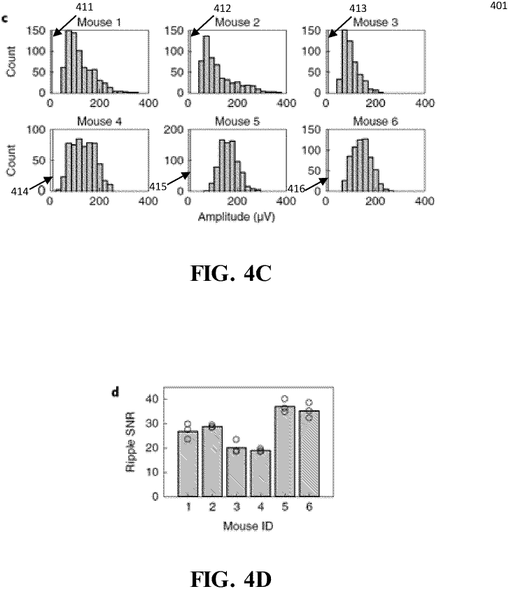

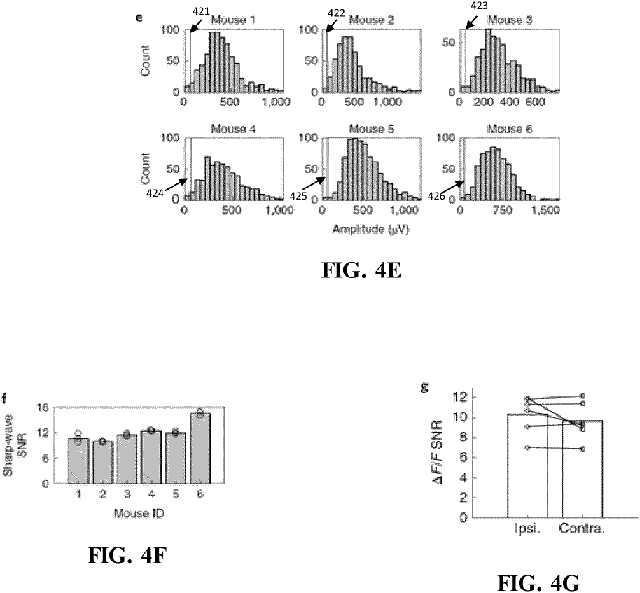

FIGS. 4A-4G show signal-to-noise ratio (SNR) for the spikes, LFPs and wide-field fluorescence. FIG. 4A shows a representative example of high-pass-filtered data from one channel showing the detection of multiple spikes and the MAD denoted by the width between two dashed lines (402, 404). FIG. 4B shows SNR of the recorded spikes in all six mice. The bar shows the mean SNR averaged over all the neurons and the error bar denotes the s.e.m. Each dot represents the spike SNR for one neuron, and “AU” indicates arbitrary units. FIG. 4C shows histogram of amplitude of the detected ripples. The line (411, 412, 413, 414, 415, 416) shows the MAD of the ripple-range LFPs (120-250 Hz). FIG. 4D shows mean SNR for the ripples detected in all six mice. Each dot represents the mean SNR of the ripples recorded in one recording channel. FIG. 4E shows histogram of amplitude of the sharp waves during SWR. The line (421, 422, 423, 424, 425, 426) shows the MAD of the sharp-wave range LFPs (5-50 Hz). FIG. 4F shows mean SNR for the sharp waves detected in all six mice. Each dot represents the mean SNR of the sharp waves recorded in one recording channel. FIG. 4G shows SNR of the ΔF/F for the cortical regions covered by the array shank (ipsilateral, Ipsi.) versus the symmetrical cortical regions on the contralateral (Contra.) side, showing a similar SNR for both cases.

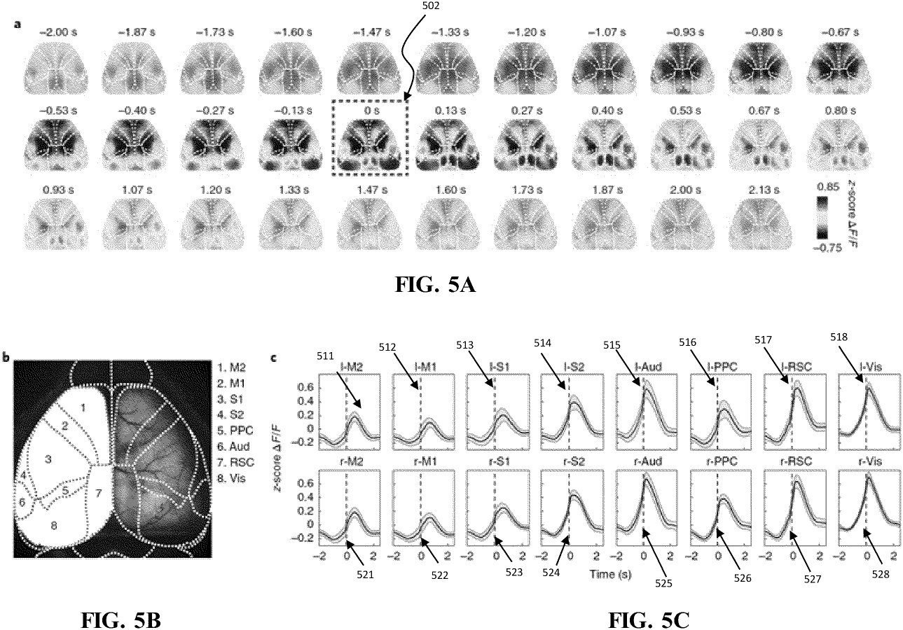

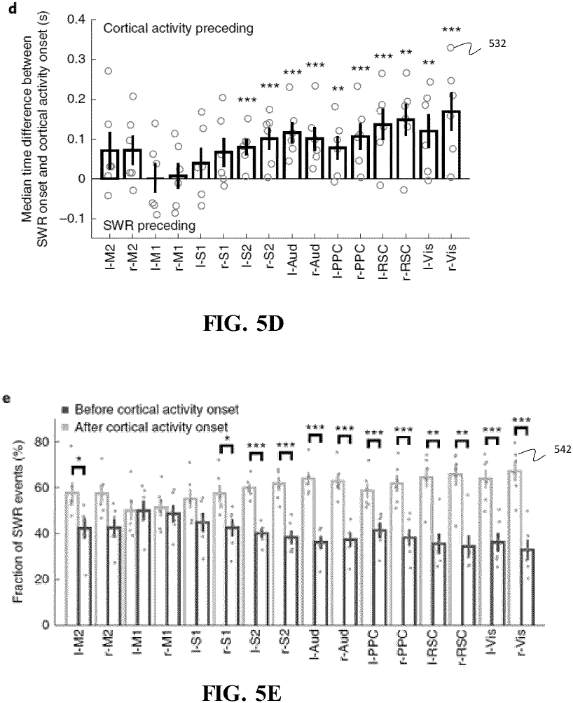

FIGS. 5A-5E show cortical activity onset tends to precede SWRs. FIG. 5A shows average cortical activity aligned to SWR onset from one example mouse. The cortex exhibits broad activation around SWRs with the cortical activity rising before SWR onset. The dashed box (502) indicates SWR onset. FIG. 5B shows identified cortical regions based on Allen Brain Atlas (“Aud” indicates auditory cortex; “M1” indicates primary motor cortex; “M2” indicates secondary motor cortex; “PPC” indicates posterior parietal cortex; “RSC” indicates retrosplenial cortex; “S1” indicates primary somatosensory cortex; “S2” indicates secondary somatosensory cortex; “Vis” indicates visual cortex). FIG. 5C shows average activity in 16 cortical regions aligned to SWR onset (mean ± s.e.m., across six animals). All cortical regions increased activity around SWRs. The dashed lines (511-518, 521-528) indicate SWR onset. FIG. 5D shows time difference of SWR onset relative to cortical activity onset (two-tailed bootstrap test, 10,000 times, Benjamini-Hochberg adjusted for a false discovery rate of 0.05, *P < 0.05, **P < 0.01, ***P < 0.001). The gray circles (532) indicate median time difference for each mouse. The time difference exhibits an anteroposterior gradient with earlier activity onset in posterior regions. All error bars are s.e.m. (n = 6 mice). The adjusted P values are 0.123, 0.050, 1.656, 1.493, 0.420, 0.050, 0, 0, 0, 0, 0.005, 0, 0, 0.001, 0 and 0. FIG. 5E shows fraction of SWR events with cortical activity onset before or after SWR onset (two-tailed bootstrap test, 10,000 times, Benjamini-Hochberg adjusted for a false discovery rate of 0.05, *P < 0.05, **P < 0.01, ***P < 0.001). The gray dots (542) indicate the fraction of SWR events before or after cortical activity onset for each mouse. From anterior to posterior regions, the fraction of SWR events with cortical activity onset leading SWRs increased. All error bars are s.e.m. (n = 6 mice). The adjusted P values are 0.016, 0.047, 1.601, 1.317, 0.319, 0.007, 0, 0, 0, 0, 0, 0, 0.003, 0.002, 0 and 0.

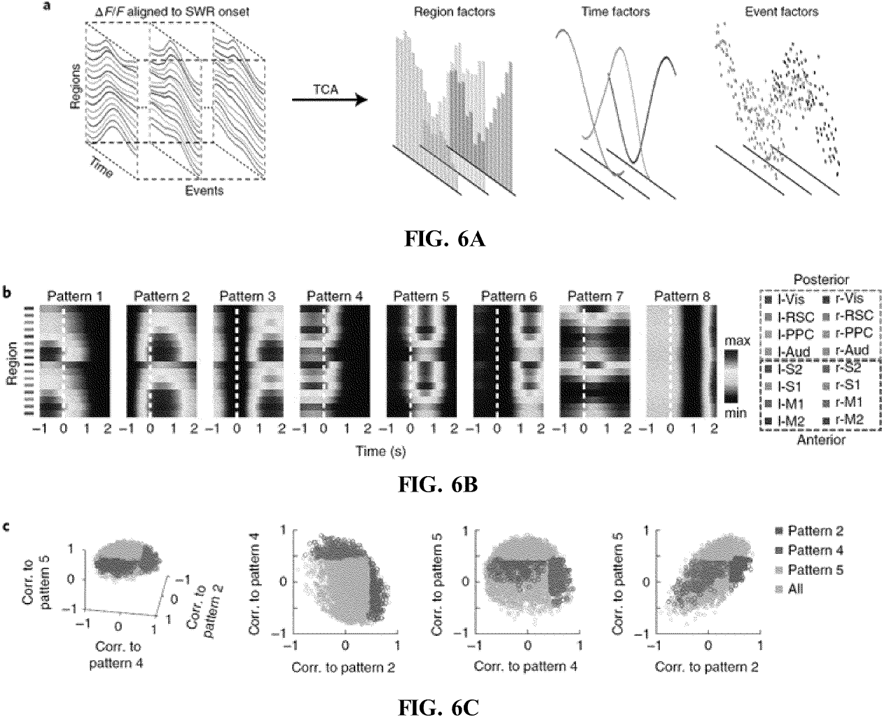

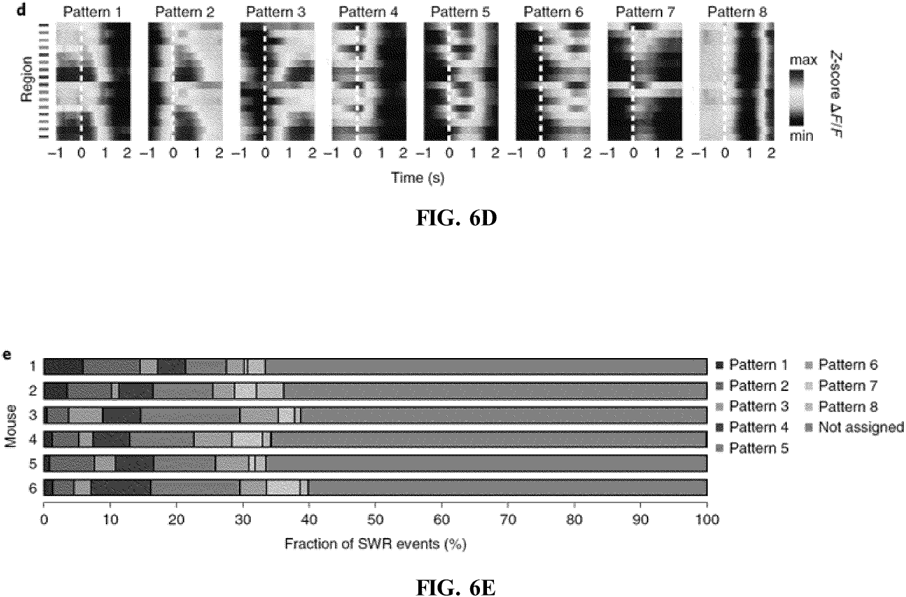

FIGS. 6A-6E show diverse SWR-associated cortical activity patterns. FIG. 6A shows schematic of the TCA algorithm (abbreviations as in FIG. 5B). The activity of 16 cortical regions during SWR events formed 3D tensors that are concatenated across mice. Using the two-stage TCA algorithm, the original data are decomposed into region, time and event factors to capture the spatiotemporal dynamics of single SWR events. FIG. 6B shows common SWR-associated cortical activity pattern templates identified across animals by the TCA algorithm. Note that patterns 1-6 exhibited activation of anterior or posterior cortical regions with three different time courses around SWR onsets. In some implementations, patterns 1-3 are defined as ‘anterior patterns’ and patterns 4-6 are defined as ‘posterior patterns’ based on the activated cortical regions. Pattern 7 is dominated by an extended activation in the visual cortex and pattern 8 shows periodic activation in all cortical regions. FIG. 6C shows correlations (Corr.) of cortical activity from single SWR events with three of the cortical activity templates. Cortical activity during single SWR events showed a continuous distribution. FIG. 6D shows SWR events, the cortical activity of which is dominated by single cortical pattern templates, are grouped separately. The figure shows the average cortical activity during SWR events assigned to each template, which closely resembled the identified cortical activity templates shown in FIG. 6B. FIG. 6E shows fraction of SWR events assigned to each cortical pattern template for all six animals. More SWR events are assigned to posterior patterns (patterns 4-6) than anterior patterns (patterns 1-3), suggesting that the posterior regions associate with SWRs more frequently than anterior regions.

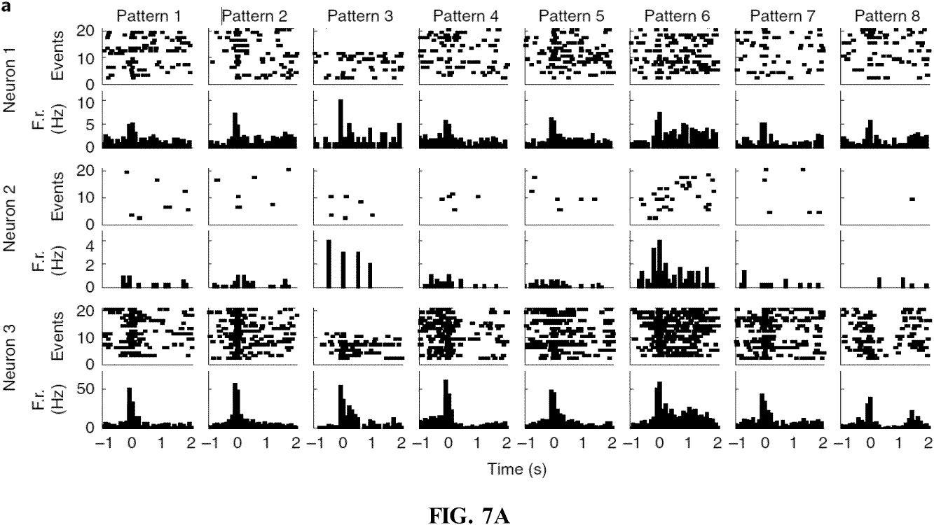

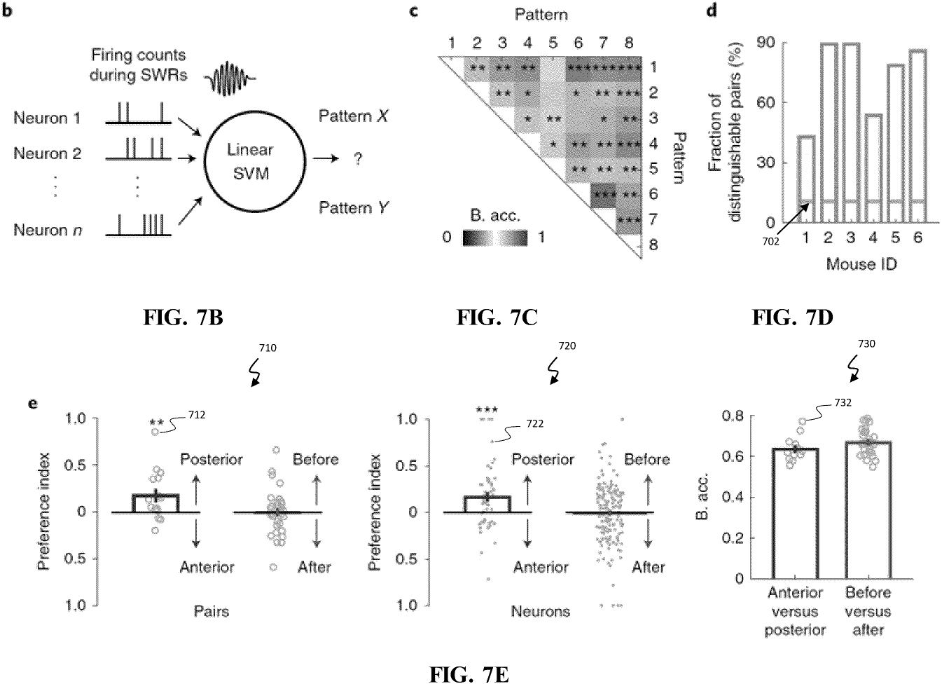

FIGS. 7A-7E show different cortical activity patterns associated with distinct hippocampal neuronal activity patterns during SWRs. FIG. 7A shows raster plots (spikes) and the peri-event time histograms of example hippocampal neurons, showing different (neurons 1 and 2) and similar (neuron 3) firing rates at SWR onset under different cortical activity patterns (“F.r.” indicates firing rate). FIG. 7B shows schematic of the decoding model. The firing counts of each hippocampal neuron during 0-100 ms relative to SWR onset are used as input features for the linear SVM to decode the cortical patterns. FIG. 7C shows decoding accuracy of all cortical pattern pairs from one example animal (mouse 2). Cortical pattern pairs that are significantly distinguishable based on hippocampal activity are marked by asterisks (shuffled 2,000 times, one tailed, *P < 0.05, **P < 0.01, ***P < 0.001; see Methods for exact P values, B. acc. indicates balanced accuracy). FIG. 7D shows fraction of distinguishable cortical pattern pairs in each animal. Across six animals, many cortical pattern pairs are distinguishable based on the hippocampal neuron activity. The gray lines (702) indicate the chance level fraction with P < 0.05 (one-sided binomial test, n = 28 pattern pairs). The P values for mice 1-6 are 2.24 × 10⁻¹⁰, 5.10 × 10⁻³², 5.10 × 10⁻³², 2.60 × 10⁻¹⁴, 9.17 × 10⁻²⁶ and 8.42 × 10⁻³⁰. FIG. 7E shows preference index and decoding accuracy between anterior (A)-posterior (P) and early (E)-late (L) pattern pairs. 710 shows preference index of discriminant hippocampal neurons between A-P pairs (pattern 1 versus 4, 2 versus 5 and 3 versus 6) or between E-L patterns (pattern 1 versus 2, 1 versus 3, 2 versus 3, 4 versus 5, 4 versus 6 and 5 versus 6). Posterior are associated with higher firing counts of discriminant neurons than the anterior patterns (two-tailed bootstrap test, 10,000 times, **P(A-P) = 0.0017, n = 15 pattern pairs) whereas no significant differences are detected between early and late patterns (P(E-L) = 0.4646, n = 33 pattern pairs). The gray circles indicate preference index averaged over all neurons for each pair within each animal. 720 shows same as left but for individual discriminant neurons (two-tailed bootstrap test, 10,000 times, ***P(A-P) = 0.0001, n = 56 neurons; P(E-L) = 0.3802, n = 160 neurons). The gray dots (712) indicate preference index of individual discriminant neurons. 730 shows decoding accuracy between A-P and E-L pairs is similar (two-tailed bootstrap test, 10,000 times, P = 0.0656, n = 15 pattern pairs for A-P, n = 33 pattern pairs for E-L). All error bars are s.e.m. The gray circles (732) indicate decoding accuracy for each pair.

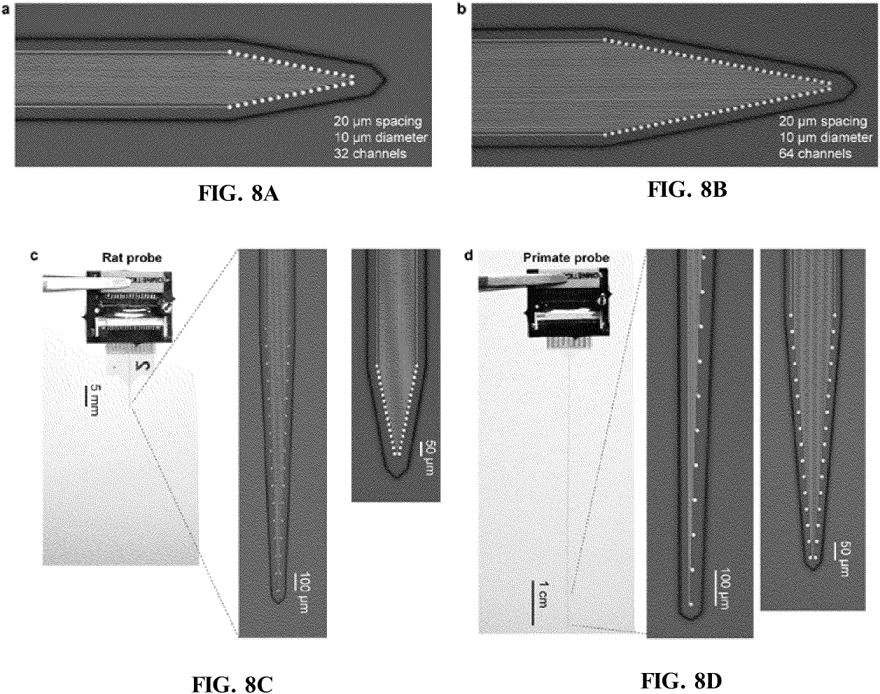

FIGS. 8A-8D show microscope pictures of different flexible, insertable, transparent microelectrode array (Neuro-FITM) probe designs. FIG. 8A shows microscope image of the recording tip of 32 channel Neuro-FITM array with 20 µm spacing. FIG. 8B shows microscope image of the recording tip of 64 channel Neuro-FITM array with 20 µm spacing. FIG. 8C shows picture of the whole probe (left), the microscope pictures of the recording tip of 32 channel Neuro-FITM array with 100 µm spacing (middle) and 20 µm spacing (right) for recording in rats. FIG. 8D is same as FIG. 8C, but for 32 channel Neuro-FITM array with 100 µm spacing and 50 µm spacing for recording in primates.

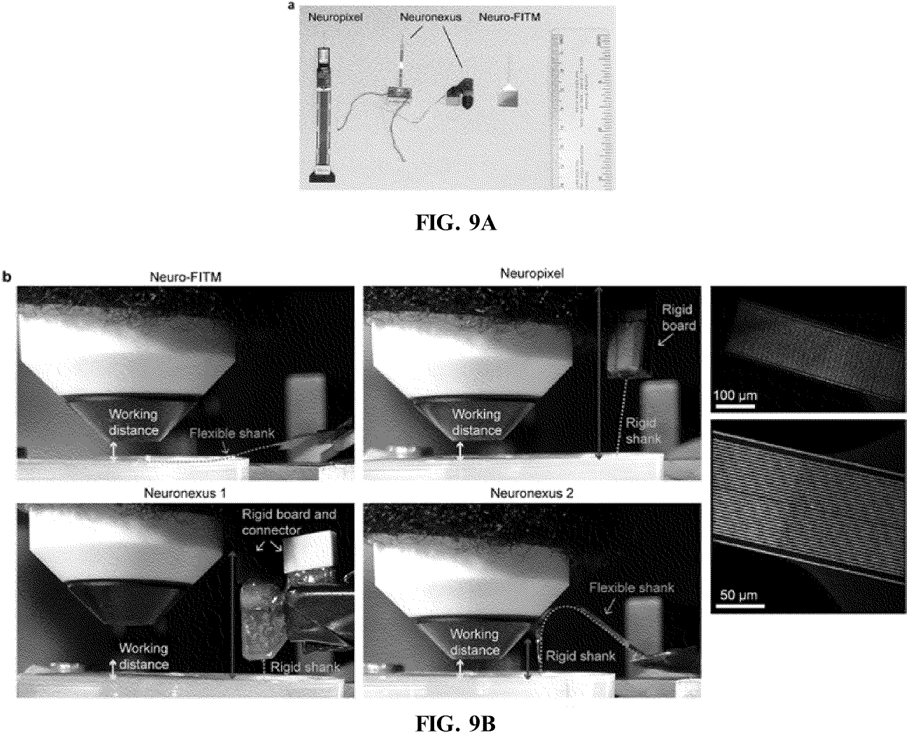

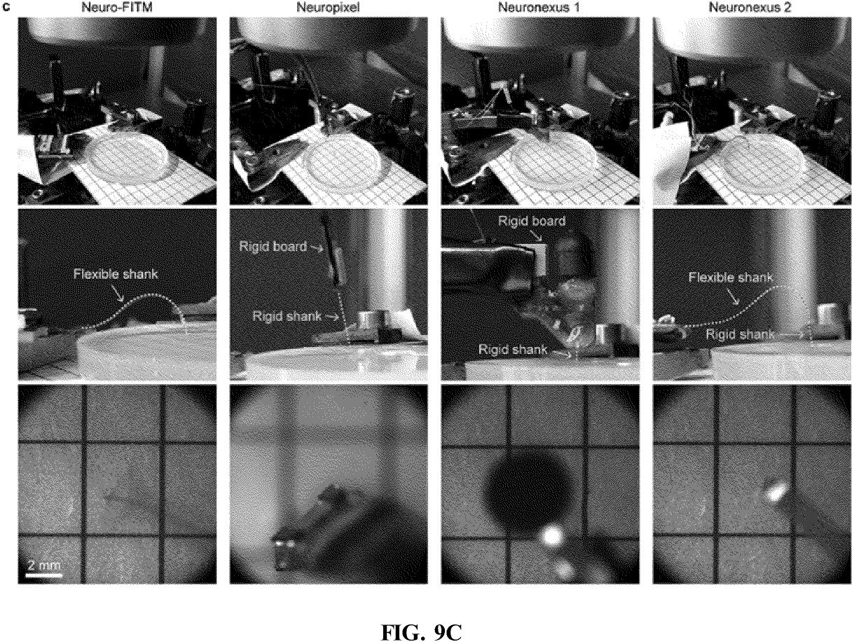

FIGS. 9A-9C show testing the multimodal recording setup using flexible, insertable, transparent microelectrode array (Neuro-FITM) and standard silicon probes under both the wide-field and 2-photon imaging systems. FIG. 9A shows a picture of the probes tested in the multimodal recording setup. FIG. 9B shows pictures of the side view under the 2-photon imaging system. Neuro-FITM can be completely bent to the side as shown with the blue dashed line. Both the Neuronexus probes and the Neuropixel probe prevent the lowering of microscope objective (total rigid part indicated by red double arrow). The right column are the 2-photon images of the array surface, showing the thin Au wires, the boundary of the array substrate, and the penetration point. FIG. 9C shows pictures of the experimental setup (top), the zoom-in side view (middle), and the field of view (bottom) under wide-field imaging system, showing the blocking of field of view (Neuronexus probes) and preventing the lowering of microscope objective (Neuropixel probe). Wide-field image shows that mostly transparent Neuro-FITM does not block the field of view or generate shadows.

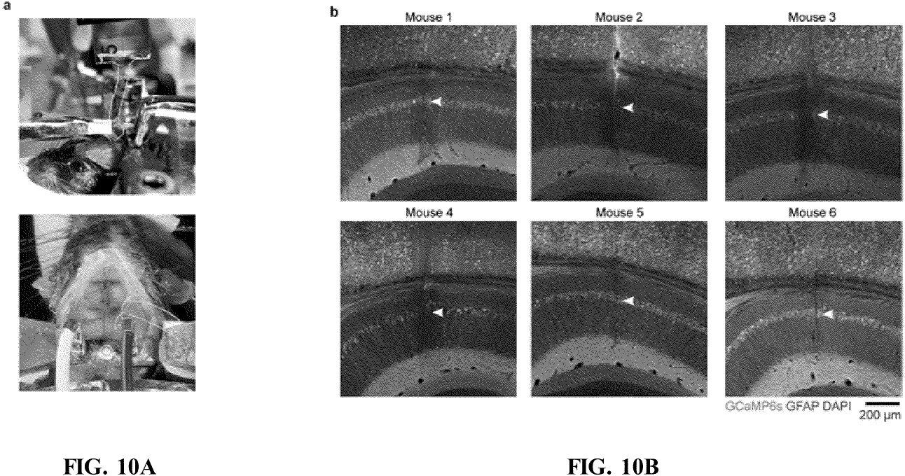

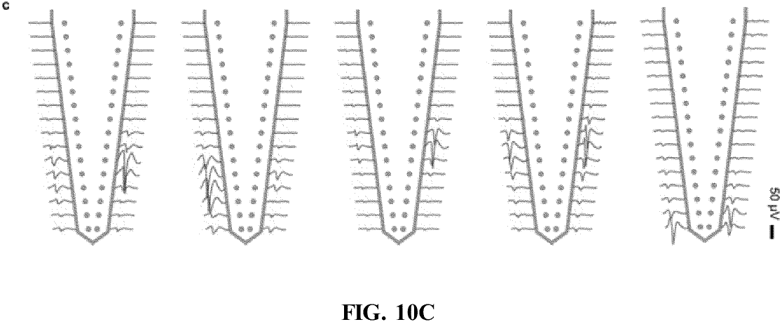

FIGS. 10A-10C show implantation of flexible, insertable, transparent microelectrode array (Neuro-FITM array) to hippocampus in in vivo experiments and the spike waveforms of example neurons. FIG. 10A shows surgical setup of array implantation in actual experiments. In some implementations, the array shank is largely invisible. The edge of the shank is marked by yellow dashed lines. FIG. 10B shows the staining results of 6 mice, showing the successful penetration to the CA1 pyramidal layer (Arrowheads: trajectory in CA1 pyramidal layer). FIG. 10C shows the spike waveforms of a few example neurons recorded from different animals. Single neurons can be detected in multiple adjacent channels, each exhibiting different waveform amplitudes.

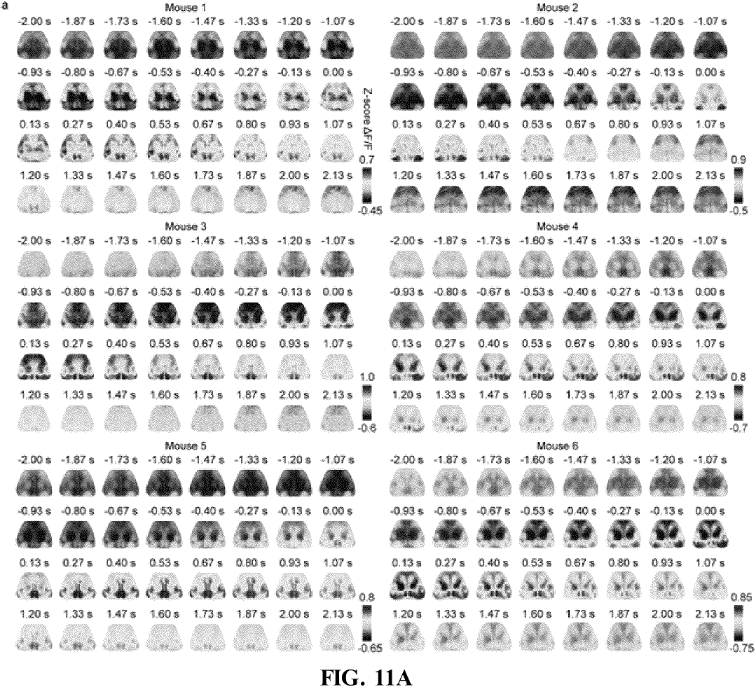

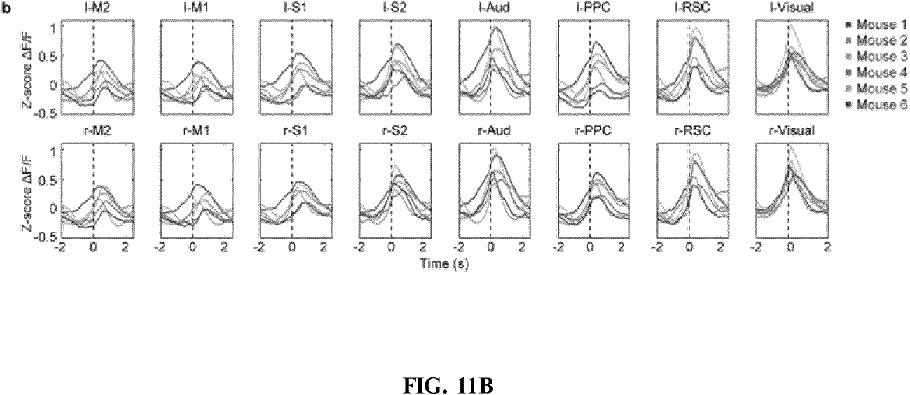

FIGS. 11A-11B show SWR-associatedlarge-scale cortical activity. FIG. 11A shows averaged cortical activity aligned to SWR onset in each animal. In all animals, the cortex exhibited broad activation around SWRs with the cortical activity rising before SWR onset. FIG. 11B shows mean activity in each cortical region aligned to SWR onset (mean ± s.e.m., across SWR events). Black dashed lines: SWR onset.

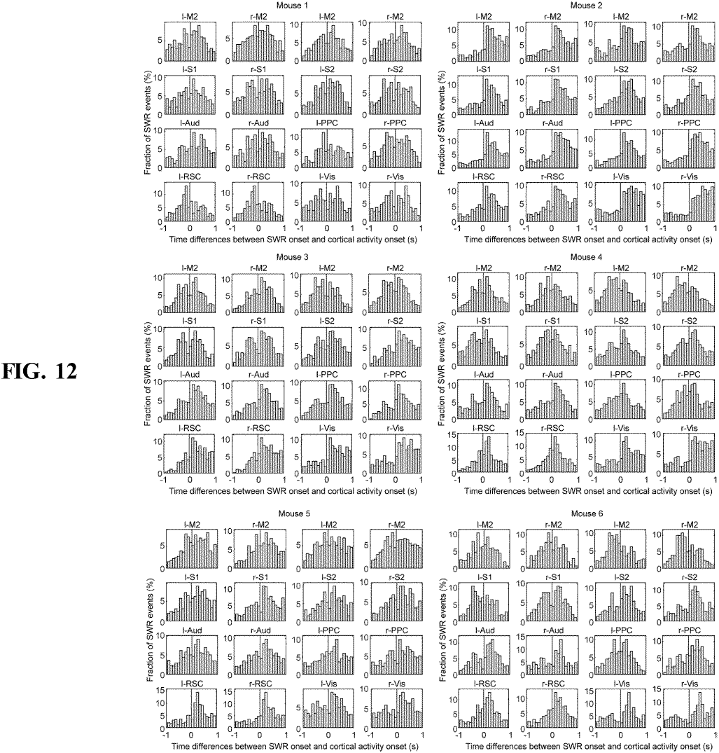

FIG. 12 shows the distribution of time differences between SWR onset and activity onset in each cortical region. The time differences (SWR onset-cortical activity onset: positive = cortex precedes SWR) formed a continuum around cortical activity onset. Note that the distribution is skewed to positive side in posterior cortical regions, suggesting cortical activity onset in posterior regions preceded SWR onset in a larger fraction of SWR events. Black lines indicate cortical activity onset.

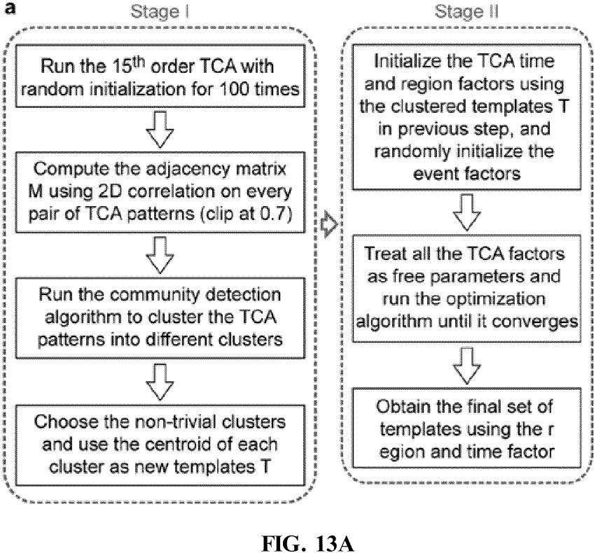

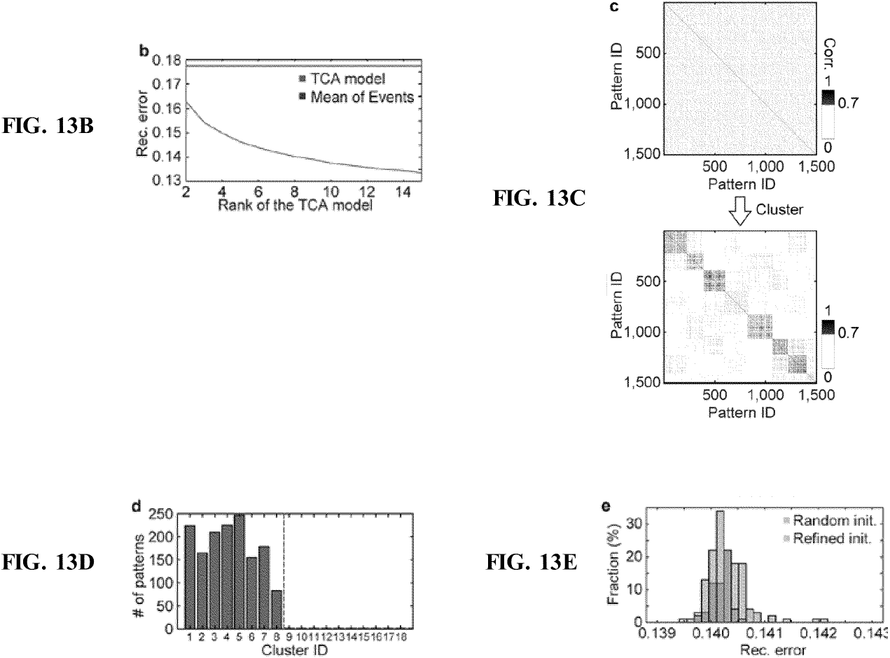

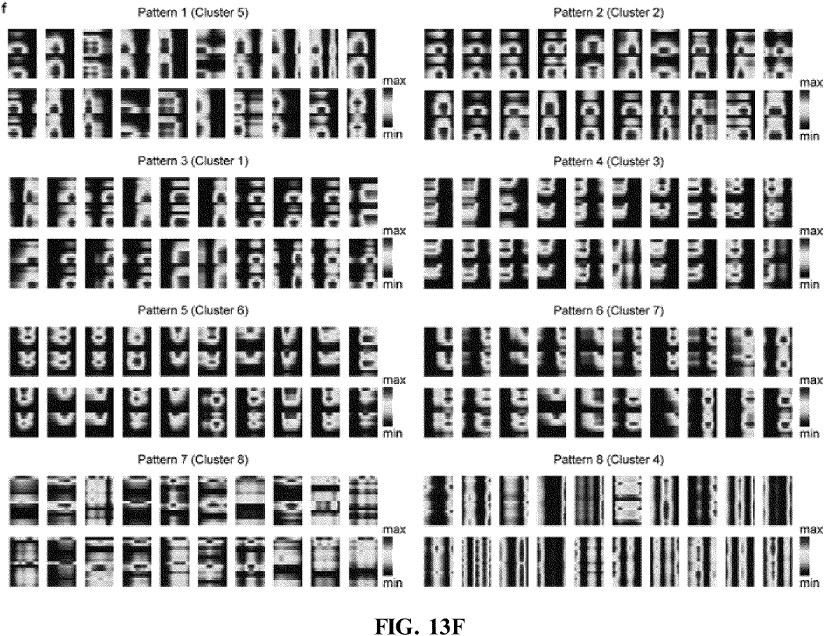

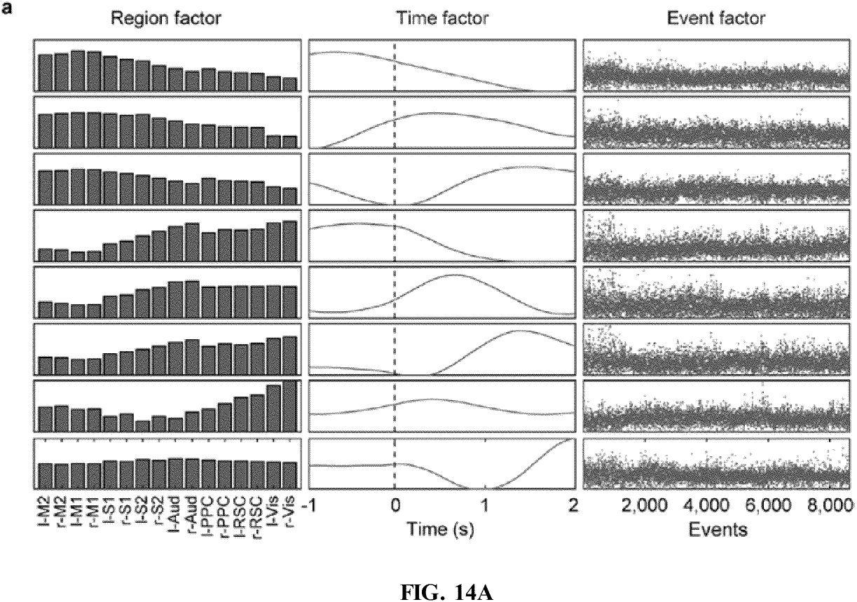

FIGS. 13A-13F show two-stage TCA algorithm. FIG. 13A shows schematic of algorithm flow. FIG. 13B shows reconstruction error (rec. error) under different ranks of TCA model. FIG. 13C shows the adjacency matrix before and after clustering. The 1,500 TCA patterns are obtained by the 100 runs of 15th order TCA with random initialization (“Corr.” indicates correlation). FIG. 13D shows number of assigned patterns in each cluster. In some implementations, only the first 8 clusters had number of assigned patterns > 1. FIG. 13E shows reconstruction error (rec. error) of the original TCA algorithm with random initialization and the two-stage TCA algorithm with refined initialization (rank = 8). The reconstruction error given by the two-stage TCA model is smaller than that of the original TCA algorithm with random initialization (two-tailed rank-sum test, P=1.38 ×10⁻¹¹, n = 100 repetitions for each algorithm), indicating that the two-stage TCA better captured the dynamics of cortical activity. FIG. 13F shows randomly selected 20 TCA patterns in each cluster for clusters 1-8. Patterns within each cluster exhibit similar spatiotemporal properties.

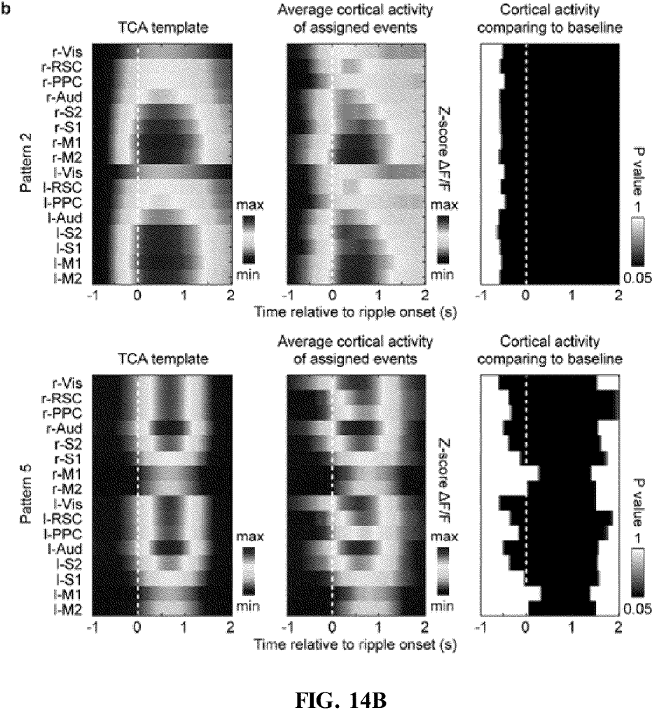

FIGS. 14A-14B show the two-stage TCA result and the cortical activation timing analysis for two patterns. FIG. 14A shows factors generated by two-stage TCA algorithm. The high-dimensional data of SWR-associated activity from 16 cortical regions is decomposed into 3 factors. The region factors and time factors describe the spatial and temporal dynamics of cortical patterns respectively and the event factors measure the weighting of a given SWR event on the established set of patterns. FIG. 14B shows cortical activation timing for pattern 2 and pattern 5. Shown in each row are the pattern template (left), the average cortical activity for the events assigned to the pattern (middle), and the P-value maps (right) for all the cortical regions at [-1 s, 2 s] time interval aligned to SWR onset, showing significantly higher activity than baseline (-1 s) for most cortical regions.

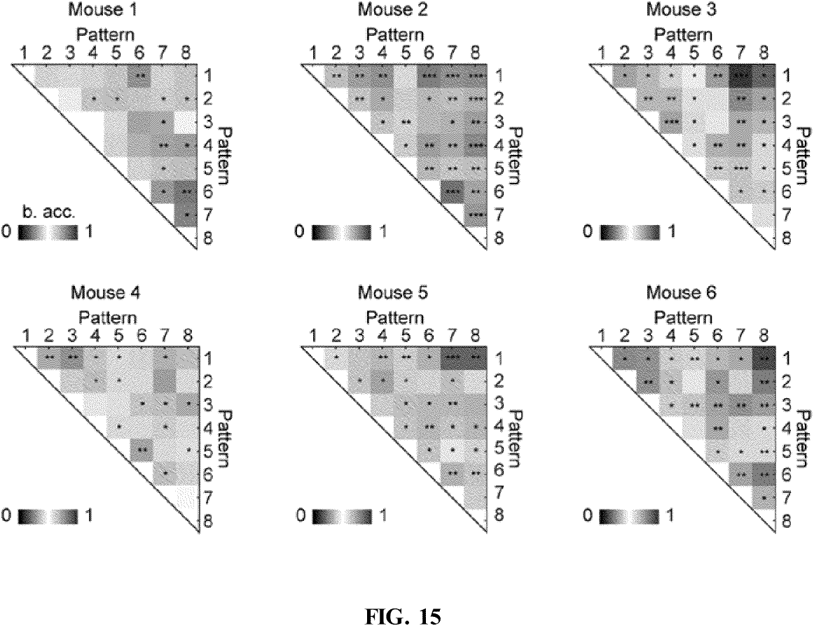

FIG. 15 shows the decoding accuracy of all cortical pattern pairs in each animal. Many cortical pattern pairs can be distinguished from each other in each animal. The distinguishable pattern pairs are marked by asterisks (shuffling 2,000 times, one-tailed, *P < 0.05, **P < 0.01, ***P < 0.001, see Methods for exact p values, “b. acc.” indicates balanced accuracy.

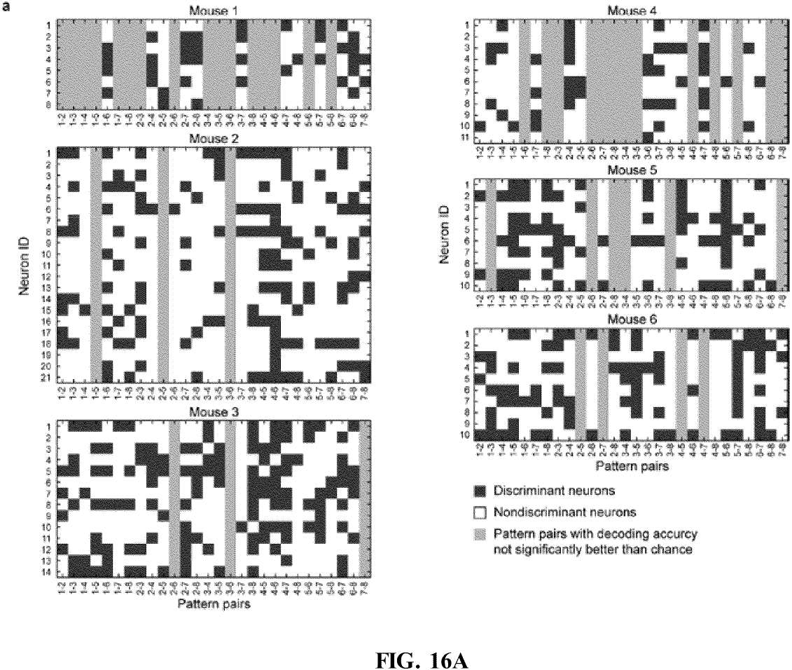

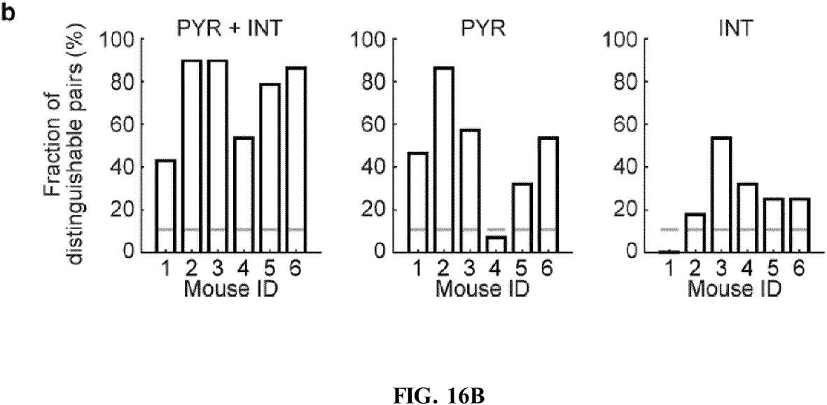

FIGS. 16A-16B show discriminant neurons in decoding cortical pattern identity and the fraction of distinguishable pairs using different neuron populations. FIG. 16A shows discriminant neurons selected by feature elimination algorithm in decoding for each pattern pair. In some implementations, the decoding often requires information from multiple hippocampal neurons, and all hippocampal neurons contributed to the decoding of some pattern pairs. FIG. 16B shows the decoding results of cortical patterns using both the PYR and INT, the PYR only, and the INT only. Gray lines indicate the chance level fraction with P < 0.05. The chance level number of decodable pattern pairs (nc) is computed from the inverse of binomial cumulative distribution with probability 0.95 (one-sided binomial test, n = 28 pattern pairs). The chance level fraction is obtained by dividing nc with n = 28, the number of pattern pairs on which decoding is performed. PYR: pyramidal neurons, INT: interneurons. For PYR + INT, the p-values for mouse 1-6 are 2.24E-10, 5.10E-32, 5.10E-32, 2.60E-14, 9.17E-26, 8.42E-30. For PYR only, the p-values for mouse 1-6 are 1.26E-11, 8.42E-30, 9.63E-16, 0.16, 5.56E-7, 2.60E-14. For INT only, the p-values for mouse 1-6 are 0.76, 0.0023, 2.60E-14, 5.56E-7, 4.92E-5, 4.92E-5.

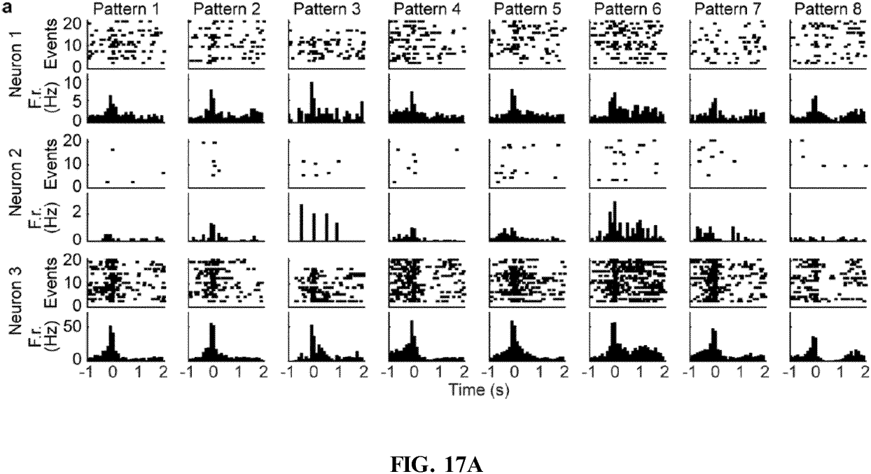

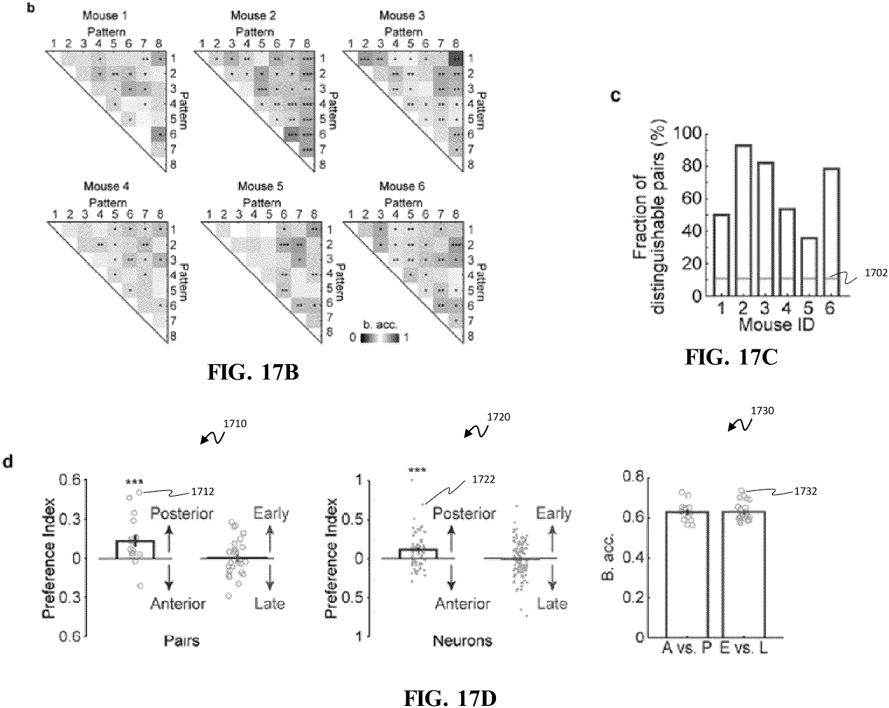

FIGS. 17A-17D show different cortical activity patterns associated with distinct hippocampal neuronal activity patterns during all SWRs. FIG. 17A shows raster plots (spikes) and the peri-event time histograms of example hippocampal neurons. FIG. 17B shows decoding accuracy of all cortical pattern pairs from all 6 animals. Cortical pattern pairs that are significantly distinguishable based on hippocampus activity are marked by asterisks (shuffled 2,000 times, one-tailed, *P < 0.05, **P < 0.01, ***P < 0.001, see Methods for exact p values, “b. acc.” indicates balanced accuracy). FIG. 17C shows fraction of distinguishable cortical pattern pairs in each animal. Gray lines (1702) indicate the chance level fraction with P < 0.05. The p-values for mouse 1-6 are 6.13×10⁻¹³, 1.99×10⁻³⁴, 1.00×10⁻²⁷, 2.60×10⁻¹⁴, 4.73 ×10⁻⁸, 9.17×10⁻²⁶, n = 28 pattern pairs. FIG. 17D shows preference index and decoding accuracy between anterior (A)-posterior (P) and early (E) - late (L) pattern pairs. 1710 shows preference index of discriminant hippocampus neurons between A-P pairs (pattern 1 vs. 4, 2 vs. 5, and 3 vs. 6) or between E-L patterns (pattern 1 vs. 2, 1 vs. 3, 2 vs. 3, 4 vs. 5, 4 vs. 6, and 5 vs. 6). Posterior patterns are associated with higher firing counts of discriminant neurons than the anterior patterns (two-tailed bootstrap test, 10,000 times, ***P(A-P)= 0.0005, n = 16 pattern pairs) while no significant differences are detected between early and late patterns (P(E-L) = 0.4380, n = 27 pattern pairs). Gray circles (1712) indicate preference index averaged overall neurons for each pair within each animal. 1720 is same as 1710 but for individual discriminant neurons (two-tailed bootstrap test, 10,000 times, ***P(A-P) = 0, n = 71 neurons, P(E-L) = 0.3591, n = 129 neurons). Gray dots (1722) indicate preference index of individual discriminant neurons. 1730 shows decoding accuracy between A-P and E-L pairs is similar (two-tailed bootstrap test, 10,000 times, P = 0.4745, n = 16 pattern pairs for A-P, n = 27 pattern pairs for E-L). All error bars are s.e.m. Gray circles (1732) indicate decoding accuracy for each pair.

The disclosed technology can be implemented in some embodiments to perform multimodal neural recordings using Neuro-FITM to uncover diverse patterns of cortical-hippocampal interactions.

Many cognitive processes require communication between the neocortex and the hippocampus. However, coordination between large-scale cortical dynamics and hippocampal activity is not well understood, partially due to the difficulty in simultaneously recording from those regions. In some embodiments of the disclosed technology, a flexible, insertable and transparent microelectrode array (Neuro-FITM) can be used to enable investigation of cortical-hippocampal coordinations during hippocampal sharp-wave ripples (SWRs). Flexibility and transparency of Neuro-FITM allow simultaneous recordings of local field potentials and neural spiking from the hippocampus during wide-field calcium imaging. These experiments revealed that diverse cortical activity patterns accompanied SWRs and, in most cases, cortical activation preceded hippocampal SWRs. In some implementations, during SWRs, different hippocampal neural population activity is associated with distinct cortical activity patterns. These results suggest that hippocampus and large-scale cortical activity interact in a selective and diverse manner during SWRs underlying various cognitive functions. The disclosed technology can be broadly applied in some embodiments to comprehensive investigations of interactions between the cortex and other subcortical structures.

Brain computations often require interactions between different cortical and subcortical structures. Understanding of these long-range interactions in the brain requires monitoring of simultaneous activity patterns across these areas. This could be achieved by simultaneous multimodal recordings combining electrophysiological recordings and large-scale functional optical imaging. However, seamless integration of optical imaging with electrophysiology is difficult with conventional microelectrodes because large probe shanks made of rigid and opaque materials can prevent lowering of the microscope objective and block the field of view of imaging. To address this issue, the disclosed technology can be implemented in some embodiments to provide a flexible, insertable, transparent microelectrode array (‘Neuro-FITM’), which can be implanted into deep cortical layers and subcortical structures. The flexible probe shank of Neuro-FITM can be bent to the side to allow lowering of the microscope objective. Optical transparency of the shank provides a clear field of view and prevents optical shadows or additional noise in optical signals. Low impedance of Neuro-FITM provides reliable recordings of local field potentials (LFPs), high-frequency oscillations and single units with a high signal-to-noise ratio (SNR).

The disclosed technology can be implemented in some embodiments to perform multimodal experiments with Neuro-FITM to investigate the coupling between the hippocampus and the cortex during SWRs. It has been suggested that hippocampal SWRs coordinate activity between the hippocampus and the cortex. Experiments with closed-loop manipulations have shown the indispensable role of SWRs in learning and memory. However, most studies focused only on a single or a few cortical regions, so little is known about the simultaneous interaction between multiple cortical regions and the hippocampus during SWRs. Furthermore, it is unclear whether the cortex is passively activated by hippocampal SWRs or whether certain cortical activity patterns can precede SWRs. Importantly, simultaneous variations across SWRs in hippocampal population activity and cortical activity patterns have not been studied. These questions could be addressed by simultaneous multimodal recordings that include electrophysiological recordings of the hippocampus and functional imaging of the cortex across large areas. In some embodiments, the flexible, insertable, transparent microelectrode array (Neuro-FITM) is implanted into the hippocampus and performed simultaneous electrophysiological recordings of SWRs and single units during wide-field calcium imaging of most of the dorsal cortex in awake, head-fixed mice. Empowered by the multimodal recording capability, the large-scale cortical activity patterns associated with SWRs on a single-event basis using tensor component analysis (TCA) exhibits a rich spatiotemporal diversity. Furthermore, by performing decoding analysis with a support vector machine (SVM), different cortical activity patterns relate to distinct activity of hippocampal neurons. In some embodiments, SWRs accompany diverse and specific interactions between the activity of the hippocampus and that of the cortex, and support the model that SWRs mediate diverse cortical-hippocampal interactions depending on the behavioral context and demand.

### Neuro-FITM Fabrication and Characterization

A flexible, insertable, transparent microelectrode array (Neuro-FITM array) implemented based on some embodiments of the disclosed technology can combine three key advantages: flexibility, transparency and shuttle-free implantation in a single probe. They are fabricated on transparent and flexible Parylene-C substrate (FIGS. 1A-1C). Briefly, a polydimethylglutarimide sacrificial layer is spin-coated on a silicon wafer. A 14-µm-thick Parylene-C layer is deposited with the chemical-vapor deposition method. Then, 5-nm Cr and 100-nm Au are deposited with sputtering and patterned with photolithography and wet etching. A 2-µm-thick Parylene-C layer is deposited as the encapsulation layer (FIG. 1C). Electrode openings are patterned with photolithography and oxygen plasma etching. The profile of the probe is defined with photolithography and oxygen reactive ion etching (FIGS. 1D-1F). Neuro-FITM arrays can be fabricated in various configurations depending on the specific needs of the experiments. The Neuro-FITM probe shown in FIGS. 1A-1N is designed to record hippocampal LFPs and units during optical imaging. The width of the array is 50 µm at the tip, whereas the shank is tapered up to a maximum width of 170 µm at the top. The array consists of 32 circular recording electrodes, each with a diameter of 10 µm connected to 2-µm-wide wires. The scanning electron microscope (SEM) images show the profile of the probe and well-defined electrode openings (FIGS. 1D-1F). The disclosed technology can be implemented in some embodiments to fabricate several different configurations of flexible, insertable, transparent microelectrode array (Neuro-FITM), including probes with smaller electrode spacing (20 µm) for potential use in a tetrode configuration (FIG. 8A), probes with a higher channel count (64 channels per shank; FIG. 8B), and probes with longer shanks to allow recording from deeper structures of the brain or to use in rats (FIG. 8C) and primates (FIG. 8D).

Reducing the electrode impedance is important to minimize the electrical noise, particularly for single-unit recordings. To achieve low impedance, platinum nanoparticles (PtNPs) are deposited on to 10-µm Au electrodes of Neuro-FITM probes (FIGS. 1G-1I). The electrode impedance can be controlled as a function of PtNP deposition time (FIG. 1J) and the size of the PtNP increases as the deposition time increases. The largest grains of PtNPs are about 500 nm in diameter for 180 s of deposition time. Electrochemical impedance spectroscopy (EIS) results show that the impedance of the Neuro-FITM electrodes is reduced by ~16× (FIG. 1K) as a result of PtNP deposition. Cyclic voltammetry (CV) measurements confirm that the PtNPs are actively engaged in the redox processes at the electrochemical interface (FIG. 1L). The impedance of the 10-µm-diameter electrodes is ~150 kΩ at 1 kHz, similar to those of the Neuropixel probes (~150 kQ) even though the surface area (78.5 µm²) is half the size (Neuropixel = 144 µm²). Considering the impedance is inversely proportional to the electrode area, the impedance of Neuro-FITM electrodes is effectively two times smaller than the Neuropixel probes. The disclosed technology can be implemented in some embodiments to investigate the effect of impedance reduction on recording noise. FIG. 1M shows recorded electrical noise as a function of electrode impedance, varied by controlling PtNP deposition time. Neuro-FITM electrodes exhibit sufficiently low noise (10 µV) for reliable detection and sorting of single units.

Optical transparency is important for seamless integration of electrophysiological recordings and optical imaging in multimodal experiments. The flexible, insertable, transparent microelectrode array (Neuro-FITM) implemented based on some embodiments has the optical transparency. The transmittance of the bent shank is ~95.7% and the recording tip with dense Au electrodes and interconnects shows a transmittance of ~50% (FIG. 1N). It is important to point out that, although the Au electrodes and Au wires are not transparent, the functional imaging would not be affected because: (1) Neuro-FITM is vertically implanted so that the penetrating tip of the probe does not directly block the light pathway and (2) the bent shank in the light pathway has thin Au wires, resulting in a high transmittance of ~95.7%. To better clarify the advantages of Neuro-FITM in multimodal configurations involving two-photon microscopy or wide-field imaging, the flexible, insertable, transparent microelectrode array (Neuro-FITM) can be compared with commercially available NeuroNexus and Neuropixel probes (FIGS. 9A-9C). The high flexibility of Neuro-FITM allows bending of the probe shank away to lower the microscope objective for two-photon imaging (FIG. 9B), whereas the rigid shanks of the Neuropixel and NeuroNexus probes prevent lowering of the microscope objective to its working distance. Wide-field microscope images (FIG. 9C) show that NeuroNexus and Neuropixel probes block the field of view and generate shadows. In addition, large probe shanks can also result in out-of-focus images (FIG. 9C, Neuropixel probe). Transparency of Neuro-FITM prevents blocking of the field of view and the formation of optical shadows that can obscure imaging. In addition to multiphoton imaging and wide-field imaging, the Neuro-FITM array is also compatible with other optical imaging techniques commonly used in neuroscience, including near-infrared spectroscopy and diffuse optical tomography.

### In Vivo Multimodal Recordings With Neuro-FITM

Vertical implantation of Neuro-FITM arrays is critical for not blocking the light pathway during optical imaging and minimizing implantation damage. To implant Neuro-FITM arrays vertically without using a rigid shuttle or adding a bioresorbable stiffening layer, the geometry and length of the microelectrode array can be determined by performing mechanical analysis to prevent buckling during insertion. Furthermore, the probe is designed to include additional micromanipulator pads to maximize insertion force against buckling (FIG. 2A; see Methods). Note that implantation of Neuro-FITM arrays with very long probe lengths designed for primate use (FIG. 8D) will require the aid of shuttles during the insertion step. After the insertion and successful targeting of the hippocampus (FIG. 2B), the shank of the array is bent away to the side to allow lowering of the microscope objective to its working distance and to clear the field of view of the microscope (FIG. 2A and FIG. 10A). The 2-µm-wide wires are confined to a narrow width to increase transparency of the shank and to minimize formation of shadows during imaging (FIG. 2C). To investigate the use of the flexible, insertable, transparent microelectrode array (Neuro-FITM) in in vivo multimodal experiments, the flexible, insertable, transparent microelectrode array can be implanted into the CA1 layer of hippocampus (FIG. 2B and FIG. 10B) of transgenic mice expressing GCaMP6s in most cortical excitatory neurons (CaMK2-tTA::tetO-GCaMP6s; see Methods). In some embodiments, electrophysiological recordings of CA1 and wide-field calcium imaging of the dorsal cortex can be performed simultaneously. Hippocampal SWRs are detected in multiple channels located near the CA1 pyramidal layer (FIG. 2D), with concurrent large-scale cortical dynamics monitored using wide-field calcium imaging. FIG. 2E shows representative examples of various spatial patterns of cortical activation during individual SWRs.

In addition to recordings of high-frequency SWR events, Neuro-FITM electrodes also detected spikes from multiple hippocampal neurons (12 ± 2 (mean ± s.e.m.) neurons in each animal). Most neurons could be detected in multiple adjacent channels, each exhibiting different spike amplitudes (FIG. 10C). FIG. 3A shows spike waveforms of 21 neurons recorded across different channels in three recording sessions from one animal. FIG. 3B shows the spike waveforms of all 21 neurons from the channel with the largest amplitude. Recorded neurons show stable spike waveforms across the sessions. The SNR of the electrical recordings is critical for spike detection and sorting as well as reliable detection of SWRs across different sessions. Therefore, the SNR for both unit (FIG. 4A) and LFP recordings can be investigated, adopting the method used for measuring spike SNR of Neuropixel probes. The SNR is computed as A/(0.6457 × B), where A is the maximum signal amplitude and B is the baseline taken as the median absolute deviation (MAD). The mean SNR of detected spikes is between 6 and 15 (FIG. 4B), similar to the SNR recorded by Neuropixel and other Si probes. To quantify the SNR of the LFP recordings, the SNR for ripples and sharp-wave events can be measured using the same method. The LFP signals recorded from the channels located in the pyramidal layer are bandpass filtered at the ripple frequency range (120-250 Hz) and sharp-wave frequency range (5-50 Hz), respectively. The baseline is then chosen as the MAD of the filtered signal from each channel. For each ripple event, the maximum signal amplitude is taken. The distribution of the detected amplitude and the SNR for ripples and sharp waves are shown in FIGS. 4C-4F, respectively. These results confirm that Neuro-FITM achieves high SNR for both single-unit and LFP recordings in all animals. Another important question is how the SNR of fluorescence response in wide-field imaging would be affected by the presence of Neuro-FITM electrodes. In some implementations, the SNR of the ΔF/F can be characterized to quantify whether the implanted array affects imaging quality following the existing procedure. Briefly, the onset and offset time points of each cortical activation event can be identified first. The SNR of each event is computed as the ratio between the maximum ΔF/F amplitude during activation and the s.d. of the ΔF/F fluctuation during [-1 s, 0 s] before onset. In some implementations, similar SNR for the fluorescence activity from the area covered by the Neuro-FITM shank and the corresponding area in the contralateral hemisphere (FIG. 4G), showing that Neuro-FITM does not significantly change the SNR of fluorescence signals during wide-field calcium imaging.

### Cortical Activation Onset Tends to Precede Hippocampal SWRs

The multimodal recording setup with a flexible, insertable, transparent microelectrode array (Neuro-FITM) implemented based on some embodiments provides an ideal platform to investigate the spatiotemporal properties of cortical-hippocampal interactions during SWRs. The large-scale cortical activity patterns are averaged across all SWRs. To analyze the onsets of cortical activity and SWR accurately without contamination from prior SWR events, some embodiments focus on SWRs that do not have other SWRs for at least the preceding 3 s (e.g., 4,290 ‘well-separated SWRs’ out of 8,643 SWRs). In some implementations, the onset of cortical activation averaged across SWRs preceded SWR onset by 1.33 ± 0.15 s (mean ± s.d.; FIG. 5A and FIG. 11A) whereas the peak of cortical activation occurred 0.67 ± 0.18 s (mean ± s.d.) after the SWR onset. To investigate whether different cortical regions have different activation timing relative to SWR onset, the dorsal cortex can be parcellatedinto 16 individual regions based on Allen Brain Atlas (FIG. 5B) and examined the activity of each cortical region around SWR onset. On average, all the cortical regions increased their activity around SWRs (FIG. 5C and FIG. 11B). Furthermore, the activation onset timing of cortical regions relative to the SWR onset exhibited an anteroposterior gradient, with the earlier activation of posterior cortical regions such as visual cortex, retrosplenial cortex and posterior parietal cortex (FIG. 5D and FIG. 12). Similarly, the fraction of SWR events with the activation of the cortical region leading SWR onset increased from anteriorto posterior cortical regions (FIG. 5E). Of SWRs, 93.78% had at least one cortical region with activity onset preceding the SWR onset. Taken together, in most SWR events, the cortical activation started before hippocampal SWRs, especially in posterior cortical regions.

### Distinct Patterns of Cortical Activity Around SWRs

Given multimodal recordings with Neuro-FITM show spatiotemporal variations in cortical activity from SWR event to SWR event (FIG. 2E), it is determined whether there are distinct cortical activation patterns that are reproducibly observed across subsets of the SWRs. Simultaneous wide-field imaging of the dorsal cortex and SWR recordings from the hippocampus with Neuro-FITM across many sessions generated large-scale neural datasets that can be analyzed to answer this question. To this end, a two-stage TCA is performed on the activity from all the recorded cortical regions during all SWR events, including SWRs that are and are not well separated. TCA is an unsupervised dimensionality reduction method that extracts recurring patterns in high-dimensional data (FIGS. 13A-13F) by decomposing the data into three factors (FIG. 6A). The region factors and time factors describe the spatial and temporal dynamics of cortical patterns, respectively, and the event factors measure the weighting of a given SWR event on the established set of patterns. By multiplying the region factors and time factors, eight distinct cortical activity pattern templates that are common across all animals are identified (FIG. 6B and FIG. 14A). The patterns exhibited distinct activated regions focusing on either the anterior or the posterior cortices, with patterns 1, 2 and 3 dominated by anterior regions (‘anterior patterns’) and patterns 4, 5 and 6 dominated by posterior regions (‘posterior patterns’), with different time courses relative to the SWR onset. Besides patterns 1-6 showing transient and spatially discrete activity patterns, pattern 7 is dominated by an extended activation in the visual cortex and pattern 8 showed periodic and oscillatory activation in all cortical regions. The cortical activity pattern in each SWR event could be well reconstructed as a linear sum of the eight templates weighted by the event factors (FIG. 13B).

To explore the diversity of SWR-associated cortical activity, the two-dimensional (2D) correlation between the cortical activity during individual well-separated SWR events and each of the cortical pattern templates can be measured first. The correlations for SWR events followed a continuous distribution instead of aggregating into isolated clusters (FIG. 6C), indicating that broadly distributed diverse cortical activity patterns are associated with SWRs. To examine the SWR events with divergent associated cortical activity, the analysis focuses on groups of SWR events with cortical activity that is mainly dominated by one of the cortical pattern templates (FIG. 6C, colored dots, 2D correlation >0.45). In total, ~36% of all the well-separated SWR events are assigned to one of the cortical pattern templates. The cortical activity averaged across the SWR events assigned to each cortical pattern template highly resembled the corresponding template (FIG. 6D, compare with FIG. 6B). Thus, many SWR events accompany diverse sets of reproducible cortical activity patterns. For the SWR events assigned to the two patterns with peak activity immediately after ripple onset (patterns 2 and 5), the activity onset of most cortical regions preceded ripple onset by 0.16-0.6 s (FIG. 14B). FIG. 6E shows the fraction of SWR events assigned to each pattern for all the mice. Overall, there are more SWR events associated with the posterior cortical patterns than the anterior patterns, suggesting a more frequent coupling between the hippocampus and posterior cortical regions during SWRs.

### Different Cortical Patterns Associate With Distinct Hippocampal Activity

Considering that SWR-associated cortical activity exhibited distinct patterns, it is determined whether hippocampal neuronal activity during individual SWR events is differentially modulated depending on the concurrent cortical patterns. In addition to SWRs, Neuro-FITM electrodes also detect spikes from the nearby hippocampal neurons in multimodal experiments. FIG. 7A shows three representative hippocampal neurons exhibiting selective (neurons 1 and 2) or nonselective (neuron 3) firing rates at the onsets of SWRs associated with different cortical patterns. To study the distinct modulation of hippocampal neurons during different cortical activity patterns, SVM decoding analysis is performed to examine whether cortical patterns could be discriminated based on the hippocampal population activity. SVM is a decoding technique that looks for a hyperplane to best separate the data according to their classes, while maximizing the margin between the data samples and the hyperplane. SVM has been shown to give a robust decoding performance for high-dimensional data, especially when the size of the dataset is limited. As a result of this advantage, it has been commonly used to decode stimuli and choices using neuronal activity. In some embodiments, an SVM decoder performs pairwise discrimination of cortical patterns based on hippocampal population activity. The SWR events associated with two cortical patterns are selected, and the decoder attempted to discriminate the cortical patterns using the spiking activity of the simultaneously recorded hippocampal neurons (12 ± 2 neurons in each animal; FIG. 7B). In some embodiments, the recursive feature elimination algorithm, which selected the subset of neurons in each decoder with activity that is informative about the cortical activity patterns (‘discriminant neurons’), can be used. This process is repeated for all pairs of cortical patterns. For many cortical pattern pairs, the cortical patterns could be discriminated significantly above chance based on the activity of hippocampal neurons during SWRs. FIG. 7C shows the decoding accuracy for each cortical pattern pair from one example mouse. In all six mice, a large fraction of cortical pattern pairs is distinguishable (FIG. 7D and FIG. 15). By examining the decodable cortical pattern pairs, it is found that different subsets of hippocampal neurons are discriminant for different cortical pattern pairs (FIG. 16A), and all hippocampal neurons are discriminant in at least one of the pairs. These results suggest that all hippocampal neurons are modulated differently depending on cortical activity patterns during SWRs. The decoding analysis is repeated using hippocampal pyramidal cells and interneurons separately. In some implementations, both hippocampal pyramidal cells and interneurons can decode the cortical activity pattern, indicating that both neuron types are modulated specifically during SWRs (FIG. 16B).

Given that many cortical pattern pairs could be decoded, it can be determined whether hippocampal neuron activity exhibited consistent modulations based on the different features of cortical activity patterns. To address this issue, two groups of pattern pairs are analyzed. One included pattern pairs with the same activation time course but different activated regions (anterior versus posterior, pattern 1 versus 4, 2 versus 5 and 3 versus 6), whereas the other included pattern pairs with the same activated regions but different time courses (early versus late, for example, pattern 1 versus 2 or 4 versus 5). To compare the activation levels of discriminant neurons determined by the recursive feature elimination algorithm for cortical pattern pairs (FIG. 16A), the ‘preference index’ for each neuron can be defined as the difference in the spike counts during one pattern versus the other, divided by the sum of the two (Methods). When comparing posterior with anterior patterns activated at similar timing, it can be found that posterior patterns are associated with higher firing in a majority of discriminant neurons than the anterior patterns, which is evident in a significantly positive preference index (FIG. 7E). In contrast, when comparing cortical activation of similar areas but with different timing, the general activity level of discriminant neurons did not show a significant preference for earlier versus later activation (FIG. 7E). Despite the lack of consistent difference in the general hippocampal activation level for E-L pattern pairs, their decoding accuracy is similar to that for A-P pattern pairs (FIG. 7E). In some implementations, the same decoding analysis and preference index analysis for all the ripple events, including the non-well-separated SWRs (FIGS. 17A-17D), can also be repeated. The results are qualitatively similar compared with FIGS. 7A-7E, indicating that the conclusions are generalizable across heterogeneous ripples. Taken together, these results reveal diverse associations between cortical activity patterns and hippocampal neuronal activity during SWRs. The posterior cortical activation is associated with stronger hippocampal activation in most of the hippocampal neurons. The relative timing between cortex and SWRs is associated with heterogeneous modulation of individual hippocampal neurons.

In some embodiments, a mostly transparent, bendable microelectrode array (Neuro-FITM) can be implemented to enable cortex-wide simultaneous optical imaging during electrophysiological recordings. To achieve the same goal, conventional silicon probes would have to be inserted contralaterally or horizontally, which would inevitably lead to long insertion trajectories causing additional implantation damage to the brain tissue. Furthermore, horizontal implantation will cause increased mechanical stress applied on to the thin silicon shank at the clamping point, which can lead to premature fracture of the probe. Instead, the flexible array implemented based on some embodiments may be inserted vertically to the hippocampus with the shortest trajectory, minimizing brain tissue damage. In addition, the flexible, insertable, transparent microelectrode array (Neuro-FITM) implemented based on some embodiments has up to 64 recording electrodes per shank, providing a higher spatial resolution for electrophysiology compared with other polymer-based microelectrodes used for hippocampal recordings. Given the high flexibility and small dimensions of the insertable shank of the array, the flexible microelectrode array may improve the stability of unit recordings in chronic studies.

The flexible, insertable, transparent microelectrode array (Neuro-FITM array) implemented based on some embodiments of the disclosed technology may potentially be combined with other neural technologies that further expand its applications into various neuroscience studies. For example, Neuro-FITM array could be integrated with wireless electrophysiological recording platforms for wireless data transmission, which are ideal for recordings in freely moving animals. The Neuro-FITM array could also be augmented to allow simultaneous electrophysiological recordings and manipulations of neural activity. This could be achieved by optimizing the charge injection capacity of the electrodes for electrical stimulation, or by incorporating micro-light-emitting diodes or waveguides into the device to form optoelectronic neural interfaces.

The simultaneous multimodal recordings of the hippocampal and cortical activity allowed us to characterize the cortical-hippocampal interactions during individual SWRs. In contrast to the conventional notion that cortical activity is mainly triggered by hippocampal SWRs, our findings suggest that the hippocampus and cortex exhibit bidirectional communications, with the cortical activation frequently preceding SWR onset. Furthermore, the relative timing between cortical activation and SWRs is area specific. The cortical activation could start before or after SWRs in both anterior and posterior cortical regions, whereas the activation of posterior cortical regions precedes SWRs more frequently than that of anterior regions. An embodiment in nonhuman primates performed simultaneous functional magnetic resonance imaging (fMRI) recordings of the whole brain and electrophysiological recordings of the hippocampus, and showed that the activation of several cortical regions can, on average, precede hippocampal SWRs. However, the SNR of fMRI limited their analysis to the average activity across SWRs and prevented the analysis of the diversity of cortical activity during individual SWRs. The approach adopted in some embodiments of the disclosed technology can achieve a sufficient SNR to perform single-event analyses across large recording areas to uncover the remarkable and coordinated diversity of cortical and hippocampal activity during SWRs. The activation of different cortical regions with different timing relative to SWR onset forms distinct cortical activity patterns from SWR to SWR. Importantly, these cortical activity patterns differentially associate with the hippocampal neuronal activity, which indicated that these patterns are not merely random fluctuation but that there is, rather, a predictable relationship of cortical activity patterns with hippocampal neuron populations, indicative of large-scale neuron assemblies that span the hippocampus and cortex.

The interaction between hippocampus and single brain regions under different behavioral states has been extensively studied. For example, it has been reported that awake SWRs are accompanied by the reactivation of neurons in the prefrontal cortex, suggesting that the awake SWRs played important roles in memory retrieval. On the other hand, the existence of a bidirectional loop between the hippocampus and the auditory cortex, which could play a role in memory consolidation, is also demonstrated. A recent study showed that, on a larger scale, the coupling between hippocampal ripples and ripples in association cortices becomes stronger after spatial learning, suggesting a closer communication between the hippocampus and association cortices during memory transfer. The hippocampus encodes a variety of information including spatial, sensory and reward. The broad and diverse activation of cortical regions observed during hippocampal SWRs may reflect a specific binding of distinct types of information encoded in the hippocampus and the relevant cortical regions through different anatomical connections. The diversity of cortical-hippocampal interactions around SWRs suggests that the hippocampus and cortex can communicate through multiple information streams based on contexts and cognitive processes. Future studies should uncover how such cortical-hippocampal interaction is dynamically shaped when the animals are experiencing different task contexts or under different behavioral states.

### Methods

Array design and measurement. The Neuro-FITM array has 32 or 64 electrodes with a flexible shank (FIGS. 1A-1B and FIGS. 8A-8D). The electrodes are aligned in two rows that are 20 µm apart from either edge of the probe. The diameter of each electrode is 10 µm and the spacing between adjacent electrodes is 50 or 20 µm. For the electrode designed to record in mouse hippocampus, the distance between the top and bottom electrodes is 750 µm, which is long enough to record from multiple depths of the CA1 region in the dorsal-ventral axis. The microelectrode array consists of a 1.55-mm probe and a 1.9-cm transparent flexible shank, connecting the electrodes to the ZIF connector. To determine the optimal length of the shank for shuttle-free insertion, a mechanical analysis can be performed as shown in equation (1), where w = 170 µm, t = 16 µm, L and E = 3.2 GPa are the width, thickness, length and Young’s modulus of the shank. The maximum force a probe can uphold without buckling is inversely proportional to the square of its length. As the insertion force F required to penetrate brain tissue is commonly accepted to be 1 mN, it can be estimated that the length of the probe must be shorter than 1.9 mm. Therefore, the length of the probe can be 1.8 mm, which is long enough to target the CA1 region of the mouse hippocampus, yet short enough to prevent buckling during insertion.

$${F}_{\text{BF}} = \frac{{π}^{2} E w {t}^{3}}{5.88 {L}^{2}}$$

All electrochemical characterizations are performed with potentiostat in 0.01 M phosphate-buffered saline. To measure the EIS and CV, a three-electrode configuration can be used, where the Ag/AgCl (gauge 25) served as the reference electrode, and Pt (gauge 25) as the counter electrode. During EIS, the applied AC voltage is 20 mV, with frequency ranging from 100 kHz to 1 Hz at open circuit potential. EIS of one representative array can be performed and the mean and s.d. are shown in FIG. 1K. During CV, the applied voltage between the PtNP/Au electrodes and the Ag/AgCl ones ranged from -0.9 V to 1 V (FIG. 1L). To stabilize the electrode/electrolyte interface, CV of a representative channel can be performed. During the measurement of CV and EIS, a customized Faraday cage can be used to shield from the 60-Hz powerline contamination and other electromagnetic noises.

Animals. Mice are group housed in disposable plastic cages with standard bedding in a room with a reversed light cycle (12 h: 12 h). Temperatures and humidity ranged from 18° C. to 23° C. and 40% to 60%, respectively. Experiments are performed during the dark period. Both male and female healthy adult mice (6 weeks or older) are used.

Surgery, multimodal experiments and data acquisition. Adult mice (6 weeks or older) are anesthetized with 1-2% isoflurane and injected with enrofloxacin (10 mg kg⁻¹) and buprenorphine (0.1 mg kg⁻¹) subcutaneously. A circular piece of scalp is removed to expose the skull. After cleaning the underlying bone using a surgical blade, a customized head-bar is implanted on to the exposed skull over the cerebellum (~1 mm posterior to lambda) with cyanoacrylate glue and cemented with dental acrylic. Two stainless-steel wires are implanted into the cerebellum as ground/reference. The exposed skull is covered with cyanoacrylate glue applied several times. After cyanoacrylate glue formed a solid layer, a craniotomy (~0.5 mm in diameter, ~1.5-1.7 mm lateral and ~2.1-2.3 mm posterior to bregma) is made at the right hemisphere for microelectrode array insertion and the dura over the exposed brain surface is carefully removed. The microelectrode array is connected to the amplifier board first and held by a customized electrode holder attached to a micromanipulator. The array is inserted at ~45 µm s⁻¹. Once inserted, the array is secured to the skull with a tissue adhesive. After the adhesive becomes solid, the array is carefully released from the electrode holder and the exposed part of the array shank is bent to the right side of the animal. The amplifier board is fixed on to the right head-bar clamp arm on the stage (FIG. 2A and FIG. 10A). Animals are fully awake before recordings. SWRs and spikes in multiple recording channels are recorded. To quantify the accuracy of array implantation, the distance between the target location and the actual location of the tip of the array is measured based on the staining results (FIG. 10B). In some implementations, the distance is 100 ± 33 µm in the medial-lateral direction, 113 ± 18 µm in the anteroposterior direction and 87 ± 24 µm in the vertical direction.

The wide-field calcium imaging is performed using a commercial fluorescence microscope (objective lens (1 ×, 0.25 numerical aperture)) and a CMOS camera through the intact skull as previously described. Images are acquired using an imaging application.

The microelectrode array is attached to a customized connector board that routes the electrical signals to an amplifier system. Electrophysiological recordings are performed using the amplifier system. The sampling rate is 30 kHz. For each animal, all recording sessions are on the same day with a 5- to 10-min interval between sessions. In some implementations, six mice are recorded, each having two to three sessions. The length of each session can be 1 hour.

Immunohistochemistry. The microelectrode array is left in the brain for 4-5 weeks before perfusion to allow glial scar formation, which is a good indication of the array location. The mice are anesthetized and perfused transcardially with 4% paraformaldehyde. Brains are then cryoprotected in a 30% sucrose solution overnight. Then, 50-mm coronal sections are cut with a microtome and blocked in a solution consisting of 4% normal donkey serum, 1% bovine serum albumin and 0.3% Triton X-100 in phosphate-buffered saline for 1 h at room temperature. They are then incubated overnight at 4° C. with primary antibodies (1:1,000 chicken anti-green fluorescent protein (GFP); 1:400 goat anti-glial fibrillary acidic protein (GFAP)) diluted in the blocking solution. After washing, sections are then incubated in Alexa Fluor-conjugated secondary antibodies (1:1,000 anti-chicken 488; 1:1,000 anti-goat 594) for 2 h at room temperature. Slices are then mounted with a mounting medium for DAPI staining and imaged using a fluorescence microscope (FIG. 2B and FIG. 10B).

SWR detection, spike sorting and ΔF/F processing. The detection of SWRs is performed using the following procedures. The raw LFP signals from the channels near CA1 pyramidal layers are bandpass filtered at 100-200 Hz (eighth-order Butterworth filter) in both forward and reverse directions to prevent phase distortion. Hilbert’s transform is then used to obtain the envelope of the ripple-band signals. To detect the potential SWR events, a threshold can be set to 2-3 s.d.s above the mean. Once the ripple-band envelope crossed the threshold, one candidate SWR event is labeled. The start and end times of this candidate SWR event are then defined as the times when the envelope just passed or returned back to the mean level. Between the start and end times, if the peak amplitude of the signal envelope further exceeded 4-6 s.d.s above the mean, then an SWR event is finally identified. In some implementations, only SWR events with a duration >20 ms can be considered.

The spike sorting is performed with a spike sorting application and the output results are followed by manual curation. The recording sessions from the same day are pooled before the spike sorting to identify the same neurons across sessions. The LFP data are first high-pass filtered at 250 Hz (third-order Butterworth filter) and whitened to remove the correlation between nearby channels. Then the spike sorting application algorithm identifies the best templates and the putative clusters of neurons, along with their spike timing and amplitudes. These preliminary results are further manually refined by merging the same neurons, splitting different neurons and labeling low-amplitude inseparable spikes as multi-unit activities. Finally, the hippocampus pyramidal cells and interneurons are classified based on the firing rates and the asymmetry of the spike waveforms.

To obtain the ΔF/F time series from the wide-field calcium imaging data, images of 512 × 512 pixels² are first down-sampled to 128 × 128 pixels². For each pixel, time-varying baseline fluorescence (F) is estimated for a given time point as the 10th percentile value >180 s around it. For the start and end of each imaging block, the following and preceding 90-s windows are used to determine the baseline, respectively. The raw ΔF/F of each pixel is z-score normalized. Corrections can be made for hemodynamic contamination following published procedures. In some implementations, principal component analysis (PCA) is performed, followed by independent component analysis (ICA) on z-score-normalized ΔF/F to extract hemodynamic components from the total signal. In some implementations, PCA is first performed and the top 50 PCs, which explained ~95% variance of the data, are preserved. Then the spatial ICA is performed over the top 50 PCs to generate 50 spatially independent modules. Finally, the modules containing the vasculature activities are excluded and the reconstruction of cortical activity is done with the remaining modules. Different numbers of components (20, 40, 50, 150 and 200) preserved in PCA/ICA analysis can be screened and, using 50 components, separation of hemodynamic and neural signal can be performed. To obtain the ΔF/F of each cortical region, the dorsal cortex is manually parcellated into individual regions based on the Allen Brain Atlas (FIG. 5b) and the ΔF/F time series is computed as the mean of the pixel values within each cortical region.

The time delay between cortical activation and SWRs. For the analysis of the timing of SWR onset and the onset of dorsal cortex activity averaged across SWR events (FIG. 5A and FIGS. 11A-11B), only the well-separated SWRs that did not have any preceding SWR events for at least 3 s can be included to prevent potential contamination from the tail of cortical activity associated with preceding SWRs. The onset timing of the event-averaged cortical activity is defined as the earliest activity onset across 16 cortical regions. For each region, using the ΔF/F at -2 s relative to SWR onset as the baseline, rank-sum tests may be performed at each frame between -2 s and 2 s relative to SWR onset. The activity onset time for each cortical region is defined as the time when its ΔF/F is significantly higher (P < 0.05) than the baseline for at least three consecutive frames. The mean onset time is computed by first averaging across sessions within animals and later averaging across animals. The peak time of event-averaged cortical activity is defined as the time when cortical activity averaged across 16 regions reach the maximum value. The mean peak time is computed by first averaging across sessions within animals and then averaging across animals.

For the analysis of timing between SWR onset and the activity onset of each cortical region during individual SWRs (FIGS. 5D and 5E and FIG. 12), the analysis may focus on well-separated SWR events. The activity onset of each cortical region is identified as previously described. In some implementations, the derivative of the smoothed ΔF/F traces (loess, 1-s window) can be computed and the inactive segments can be defined as the periods with the derivative within 1 s.d. of the whole derivative trace. Then ΔF/F events can be defined as the periods when the derivative exceeded the 1 s.d. of the inactive period. For each event, the onset time is first estimated as the time when the derivative exceeded the 1 s.d. criterion, and the offset time is estimated as the time when the derivative dropped to <0 for the first time after the onset. To further refine the onset time, for each event, the baseline Δ/F/F is defined as the value at the first time point when the derivative is >0 before the offset time, and ΔF/F noise level is defined as the mean of the absolute difference between the raw and smoothed ΔF/F traces. The onset is further refined as the last time point before the offset time when the ΔF/F value is within the noise level from the baseline ΔF/F.

After identifying the activity onset of each cortical region, the timing of each SWR onset relative to the activity onset of each region is determined using the following procedures. For each SWR onset, the slope of the instantaneous ΔF/F traces of one region is first examined. If the ΔF/F is rising, we loop backward in time frame by frame until reaching-1 s before the SWR onset. If a cortical activity onset is detected within this time interval, this SWR event is labeled as occurring after the cortical activity onset. On the other hand, if the ΔF/F is not rising, we loop forward in time frame by frame until reaching +1 s after the SWR onset. If a cortical activity onset is detected within this time interval, this SWR event is labeled as occurring before the cortical activity onset. The above procedure is done for every well-separated SWR and all the cortical regions.

Two-stage TCA algorithm. To prepare the data for the TCA algorithm, the preprocessing procedures described below are performed. The ΔF/F traces in each cortical region are z-score normalized within each recording session. For each SWR event, the 3-s ΔF/F traces (1 s before SWR onset, 2 s after) from 16 cortical regions can be used to construct a 2D data matrix (region × time). Then the 2D data matrices from all the SWR events are concatenated to form a three-dimensional (3D) data tensor (region × time × event). Finally, the data tensors from all the six mice are concatenated along the event dimension to form a big data tensor (FIG. 6A).

The TCA has been demonstrated to be effective in discovering the low-dimensional dynamics of neural activity. However, as the original algorithm did not guarantee achieving the global optimum, the results could vary from run to run. To achieve reliable results, the disclosed technology can be implemented in some embodiments to provide a two-stage TCA algorithm, which includes a pre-clustering step to alleviate the variations from individual runs. The detailed procedure is shown in FIG. 13A. The first stage of the algorithm includes fitting a TCA model with a sufficiently high rank order. A tensor toolbox may be used to perform TCA decomposition. To determine this rank order, the disclosed technology can be implemented in some embodiments to fit multiple TCA models with rank 2-15 and examine the reconstruction error of each TCA model. The reconstruction error starts to show diminishing returns toward rank 15 (FIG. 13B), and thus rank 15 is selected for the initial TCA and performed 100 times. Each result gave a slightly different decomposition of the original high-dimensional data. To capture the underlying dynamics that are common and consistent in most TCA results, clustering of the 1,500 TCA spatiotemporal patterns can be performed by computing the similarity matrix using 2D correlation. Then the community detection algorithm is performed with the community detection toolbox to identify the clusters. As shown in the sorted similarity matrix (FIG. 13C), eight different clusters of TCA patterns are identified. The number of patterns assigned to each cluster is shown in FIG. 13D. Examples of randomly chosen patterns assigned to each cluster are shown in FIG. 13F. The second stage of the TCA algorithm can use the centroids of eight clusters identified from the first stage to initialize the region and time factors, leaving all the event factors randomly initialized. Then the TCA optimization algorithm cab be performed as before until it converged to obtain the final set of TCA factors (FIG. 14A). Compared with the original TCA algorithm, the two-stage TCA algorithm implemented based on some embodiments of the disclosed technology can give significantly lower reconstruction error (P = 1.38 × 10⁻¹¹, FIG. 13E).

Cortical pattern assignment. To assign the cortical activity pattern of each SWR event to one of the eight spatiotemporal templates (FIG. 6B), the 2D correlation between the z-score-normalized ΔF/F traces and each template can be computed. If the correlation value for one pattern is higher than a threshold (0.45; FIGS. 6c-e), the SWR event is assigned to that pattern. If one SWR event is assigned to multiple patterns, that SWR event is excluded.

The algorithm for pairwise discrimination of the cortical patterns. To discriminate the cortical patterns based on hippocampal activity, the SVM can be used. The hippocampal neuron firing counts during 0-100 ms relative to SWR onset are used as input features for the SVM algorithm. As the numbers of SWR events assigned to each cortical pattern template are often unbalanced (FIG. 6E), the misclassification costs can be modified to be inversely proportional to the sample frequencies of the two pattern types in each pair, N1 and N2. Therefore, misclassifying pattern type 1 as pattern type 2 has cost N2/(N1 + N2), whereas misclassifying pattern type 2 as pattern type 1 has cost N⅟(N1 +N2). Also, to measure the decoding performance, balanced accuracy can be used instead of the accuracy, which could be misleading in the unbalanced datasets. The balanced accuracy can be defined as the average of the correct proportion for each class (e.g., cortical pattern). The recursive feature elimination can be performed to identify the discriminant neurons for each cortical pattern pair (FIGS. 16A-16B). This can be done by choosing the subset of neurons that give the highest balanced accuracy in the leave-one-out cross-validation. To evaluate whether the decoding performance for each cortical pattern pair is significantly better than chance, the cortical pattern identities are randomly shuffled 2,000 times, SVM is performed using the identified discriminant neurons and the balanced accuracy is computed in each shuffle to obtain a null distribution of it. Then the P value can be computed based on the balanced accuracy from the original dataset and the distribution of the balanced accuracy from the shuffled dataset (FIG. 7C, FIG. 17B, and FIG. 15). The exact P values associated with FIG. 7C are as follows: mouse 1: P(1-2) = 0.086, P(1-3)= 0.2815, P(1-4) = 0.1415, P(1-5) = 0.153, P(1-6) = 0.0035, P(1-7) = 0.094, P(1-8) = 0.0965, P(2-3) = 0.3365, P(2-4) = 0.0315, P(2-5) = 0.036, P(2-6) = 0.0535, P(2-7) = 0.0245, P(2-8) = 0.0425, P(3-4) = 0.5235, P(3-5) = 0.28, P(3-6) = 0.052, P(3-7) = 0.037, P(3-8) = 0.3795, P(4-5) = 0.13, P(4-6)=0.0695, P(4-7) = 0.005, P(4-8) = 0.016, P(5-6) = 0.153, P(5-7) = 0.017, P(5-8) = 0.062, P(6-7) = 0.0205, P(6-8) = 0.0025, P(7-8) = 0.0275; mouse 2: P(1-2) = 0.0035, P(1-3)=0.0045, P(1-4) = 0.004, P(1-5) = 0.0665, P(1-6)= 0, P(1-7) = 0, P(1-8) = 0, P(2-3) = 0.009, P(2-4) = 0.017, P(2-5) = 0.0525, P(2-6) = 0.0375, P(2-7) = 0.0055, P(2-8) = 0.0005, P(3-4) = 0.039, P(3-5) = 0.007, P(3-6) = 0.0545, P(3-7) = 0.035, P(3-8) = 0.0025, P(4-5)= 0.0125, P(4-6) = 0.001, P(4-7) = 0.002, P(4-8) = 0, P(5-6) = 0.0085, P(5-7) = 0.006, P(5-8) = 0.0015, P(6-7) = 0, P(6-8) = 0.001, P(7-8) = 0; mouse 3: P(1-2) = 0.0105, P(1-3) = 0.015, P(1-4) = 0.024, P(1-5) = 0.0275, P(1-6) = 0.0035, P(1-7) = 0, P(1-8) = 0.0295, P(2-3) = 0.008, P(2-4) = 0.006, P(2-5) = 0.017, P(2-6) = 0.2245, P(2-7) = 0.0015, P(2-8) = 0.0135, P(3-4) = 0.0005, P(3-5) = 0.017, P(3-6) = 0.1865, P(3-7) = 0.001, P(3-8) = 0.015, P(4-5) = 0.047, P(4-6) = 0.001, P(4-7)=0.0035, P(4-8) = 0.041, P(5-6) = 0.0035, P(5-7) = 0, P(5-8)=0.0165, P(6-7) = 0.0295, P(6-8) = 0.034, P(7-8) = 0.2295; mouse 4: P(1-2) = 0.0055, P(1-3) = 0.0085, P(1-4) = 0.023, P(1-5)=0.0135, P(1-6) = 0.054, P(1-7) = 0.0135, P(1-8) = 0.167, P(2-3) = 0.073, P(2-4) = 0.013, P(2-5) = 0.037, P(2-6) = 0.0765, P(2-7) = 0.3305, P(2-8) = 0.1825, P(3-4) = 0.25, P(3-5) = 0.0675, P(3-6) = 0.0175, P(3-7) = 0.03, P(3-8) = 0.029, P(4-5) = 0.034, P(4-6) = 0.0905, P(4-7) = 0.0375, P(4-8) = 0.0675, P(5-6) = 0.0015, P(5-7) = 0.0775, P(5-8) = 0.0285, P(6-7) = 0.046, P(6-8) = 0.094, P(7-8) = 0.39; mouse 5: P(1-2) = 0.0335, P(1-3) = 0.0755, P(1-4) = 0.009, P(1-5) = 0.0075, P(1-6) = 0.013, P(1-7) = 0, P(1-8) = 0.0055, P(2-3) = 0.0295, P(2-4) = 0.0145, P(2-5) = 0.0495, P(2-6) = 0.057, P(2-7) = 0.0215, P(2-8) = 0.1255, P(3-4) = 0.0875, P(3-5) = 0.0195, P(3-6) = 0.015, P(3-7) = 0.0095, P(3-8) = 0.0535, P(4-5) = 0.0155, P(4-6) = 0.009, P(4-7) = 0.0325, P(4-8) = 0.0245, P(5-6) = 0.0145, P(5-7) = 0.0415, P(5-8)= 0.026, P(6-7) = 0.007, P(6-8) = 0.0065, P(7-8) = 0.1315; mouse 6: P(1-2) = 0.018, P(1-3) = 0.0175, P(1-4) = 0.017, P(1-5) = 0.0065, P(1-6) = 0.046, P(1-7) = 0.013, P(1-8) = 0.001, P(2-3) = 0.007, P(2-4) = 0.0105, P(2-5) = 0.108, P(2-6) = 0.0115, P(2-7) = 0.1615, P(2-8) = 0.0025, P(3-4) = 0.0345, P(3-5) = 0.0025, P(3-6) = 0.008, P(3-7) = 0.001, P(3-8) = 0.0045, P(4-5) = 0.0835, P(4-6) = 0.0015, P(4-7) = 0.062, P(4-8) = 0.017, P(5-6) = 0.0315, P(5-7) = 0.03, P(5-8) = 0.0065, P(6-7) = 0.0055, P(6-8) = 0.0025, P(7-8) = 0.0125. The exact P values associated with Extended Data FIG. 10b are as follows: mouse 1: P(1-2) = 0.0645, P(1-3) = 0.1735, P(1-4) = 0.0315, P(1-5) = 0.057, P(1-6) = 0.128, P(1-7) = 0.008, P(1-8) = 0.027, P(2-3) = 0.1735, P(2-4) = 0.0375, P(2-5) = 0.0025, P(2-6) = 0.0205, P(2-7) = 0.0135, P(2-8) = 0.345, P(3-4) = 0.1685, P(3-5) = 0.0225, P(3-6) = 0.012, P(3-7) = 0.04, P(3-8) = 0.3775, P(4-5) = 0.01, P(4-6) = 0.3415, P(4-7) = 0.0415, P(4-8) = 0.289, P(5-6) = 0.042, P(5-7) = 0.1595, P(5-8) = 0.066, P(6-7) = 0.473, P(6-8) = 0.01, P(7-8) = 0.07; mouse 2: P(1-2) = 0.018, P(1-3)=0.034, P(1-4) = 0.007, P(1-5) = 0.114, P(1-6) = 0.0065, P(1-7) = 0.0245, P(1-8) = 0, P(2-3) = 0.0135, P(2-4) = 0.012, P(2-5) = 0.0115, P(2-6) = 0.037, P(2-7) = 0.0205, P(2-8) = 0, P(3-4) = 0.058, P(3-5) = 0, P(3-6) = 0.02, P(3-7) = 0.0345, P(3-8) = 0.0035, P(4-5) = 0.0115, P(4-6) = 0.0015, P(4-7) = 0, P(4-8) = 0, P(5-6) = 0.0465, P(5-7) = 0.009, P(5-8) = 0, P(6-7) = 0, P(6-8) = 0, P(7-8) = 0; mouse 3: P(1-2) = 0, P(1-3) = 0.001, P(1-4) = 0.0135, P(1-5) = 0.035, P(1-6) = 0.011, P(1-7)=0.0935, P(1-8) = 0.001, P(2-3) = 0.0575, P(2-4) = 0.0015, P(2-5) = 0.003, P(2-6)=0.0515, P(2-7) = 0.0045, P(2-8) = 0.0015, P(3-4) = 0.0025, P(3-5) = 0.0225, P(3-6) = 0.2895, P(3-7) = 0.0045, P(3-8) = 0.0025, P(4-5) = 0.002, P(4-6) = 0.0295, P(4-7) = 0.002, P(4-8)=0.0205, P(5-6) = 0.023, P(5-7) = 0.0055, P(5-8) = 0.01, P(6-7) = 0.088, P(6-8)=0.002, P(7-8) = 0.0355; mouse 4: P(1-2) = 0.221, P(1-3) = 0.177, P(1-4) = 0.111, P(1-5) = 0.0335, P(1-6) = 0.011, P(1-7) = 0.0175, P(1-8) = 0.0435, P(2-3) = 0.0765, P(2-4) = 0.0025, P(2-5) = 0.0205, P(2-6) = 0.0615, P(2-7) = 0.001, P(2-8) = 0.143, P(3-4) = 0.2925, P(3-5) = 0.0335, P(3-6) = 0.009, P(3-7) = 0.049, P(3-8) = 0.0335, P(4-5) = 0.0105, P(4-6) = 0.123, P(4-7) = 0.022, P(4-8) = 0.1275, P(5-6) = 0.0195, P(5-7) = 0.105, P(5-8) = 0.1305, P(6-7) = 0.0875, P(6-8) = 0.0255, P(7-8) = 0.11; mouse 5: P(1-2) = 0.085, P(1-3) = 0.627, P(1-4) = 0.1625, P(1-5) = 0.4755, P(1-6) = 0.024, P(1-7) = 0.259, P(1-8) = 0.009, P(2-3) = 0.105, P(2-4) = 0.052, P(2-5) = 0.1565, P(2-6) = 0, P(2-7) = 0.0065, P(2-8) = 0.09, P(3-4) = 0.142, P(3-5)=0.0705, P(3-6) = 0.176, P(3-7) = 0.014, P(3-8) = 0.12, P(4-5) = 0.0705, P(4-6) = 0.0015, P(4-7) = 0.2375, P(4-8) = 0.007, P(5-6) = 0.001, P(5-7) = 0.185, P(5-8) = 0.0995, P(6-7) = 0.0075, P(6-8) = 0.0105, P(7-8) = 0.115; mouse 6: P(1-2) = 0.063, P(1-3) = 0.021, P(1-4) = 0.023, P(1-5) = 0.0065, P(1-6) = 0.0995, P(1-7) = 0.013, P(1-8) = 0.1085, P(2-3) = 0.01, P(2-4) = 0.0105, P(2-5) = 0.004, P(2-6) = 0.0455, P(2-7) = 0.0925, P(2-8) = 0.0005, P(3-4) = 0.008, P(3-5) = 0.004, P(3-6) = 0.044, P(3-7) = 0.003, P(3-8) = 0.0165, P(4-5) = 0.0105, P(4-6) = 0.034, P(4-7) = 0.2415, P(4-8) = 0.077, P(5-6) = 0.011, P(5-7) = 0.0035, P(5-8) = 0.045, P(6-7) = 0.0035, P(6-8) = 0.033, P(7-8) = 0.012. Finally, to further quantify the overall decoding performance for each mouse, the fraction of distinguishable cortical pattern pairs (P < 0.05) can be computed over the cortical pattern pairs included in the analysis within each animal (FIG. 7D and FIG. 17C). To examine whether the fraction of distinguishable cortical pattern pairs in each animal is significant, the disclosed technology can be implemented in some embodiments to test against the null hypothesis that the fraction is obtained by chance. As the probability of each pattern pair being mislabeled as distinguishable is 0.05, under the null hypothesis, the number of distinguishable pairs in each mouse follows a binomial distribution where the parameter P = 0.05 and N equals the number of pattern pairs included in the analysis within each animal. Therefore, the critical number of pattern pairs, Nc, is determined as the smallest integer that makes the binomial cumulative density function >0.95. Finally, the chance level fraction is obtained as the ratio between Nc and N.

### Hippocampal Neuron Firing Rates Under Different Cortical Patterns During SWRs

To obtain the instantaneous firing rates between -1 s and 2 s relative to SWR onset for each hippocampal neuron, 100-ms time bins without overlap for each SWR event (FIG. 7A and FIG. 17A) can be used. The preference index (PI) is defined to measure whether one neuron showed higher activity for one pattern than the other (FIG. 7E and FIG. 17D). For each pattern pair (for example, pattern X and pattern Y), the preference index of one neuron is calculated using its mean firing count between 0 and 100 ms relative to SWR onset under each pattern, as shown in equation (2).

$$\text{PI} \left(X\right) = \frac{\text{Firing count} \left(X\right) - \text{Firing count} \left(Y\right)}{\text{Firing count} \left(X\right) + \text{Firing count} \left(Y\right)}$$

The early versus the late group included pattern pairs of pattern 1 versus 2, 1 versus 3, 2 versus 3, 4 versus 5, 4 versus 6 and 5 versus 6. The anterior versus posterior group can include pattern pairs of pattern 1 versus 4, 2 versus 5 and 3 versus 6. For each cortical pattern pair, the preference index at population level can be calculated by averaging across discriminant hippocampal neurons (FIG. 7E and FIG. 17D).

Statistics and reproducibility. For electrode arrays designed for recordings in mice, rats and monkeys, four electrode arrays are imaged, respectively, and example images are shown in FIG. 1 and FIGS. 8A-8D. Two animals are excluded from eight animals from recordings and analyses due to unsuccessful implantations. The six animals with successful implantations went through the same recording procedures and are all included in analyses. All statistical analyses are performed in MATLAB. Statistical tests are two tailed and significance is defined by an a pre-set to 0.05. Error bars and shaded regions surrounding line plots indicate ±s.e.m. unless otherwise noted. All the statistical tests are described in the figure legends and each test is selected based on data distributions using histograms. For FIG. 1M, a two-sided Student’s t-test can be used to test the correlation between the electrode impedance and the recording noise level. For FIGS. 5D and 5E, a two-tailed bootstrap test (10,000×) can be used to test the median time difference between SWR and cortical activity onset and the fraction of SWR events occurring before or after cortical activity onset. For FIG. 7C, the decodable pattern pair can be determined by a one-tailed shuffling test, which randomly permuted the labels of cortical patterns. For FIG. 7D, the chance level number of decodable pattern pairs (nc) can be computed from the inverse of binomial cumulative distribution with probability 0.95 and the chance level fraction can be obtained by dividing nc with n = 28, the number of pattern pairs on which decoding is performed. For FIG. 7E, a two-tailed bootstrap test (10,000 ×) can be used to determine the significance of preference index and the balanced accuracy. Multiple comparisons are corrected for using Benjamini-Hochberg corrections. Sample sizes (n) are as follows where applicable: recording sessions per animal, 2, 3, 3, 3, 2, 2; well-separated SWRs/all SWRs per animal, 530/1,245, 896/1,785, 787/1,440, 826/1,618, 673/1,365, 578/1,190; hippocampal neurons per animal, 8, 21, 14, 11, 10, 10. No statistical methods are used to predetermine sample size but the sample sizes are similar to those reported in previous publications from the lab using wide-field calcium imaging and electrophysiological recordings. In some implementations, no randomization is performed. In some implementations, randomization is not necessary because all animals undergo the same surgical and recording procedures. In some implementations, data collection and analysis are not performed blind to the conditions of the experiments.

### Decoding of Cortex-Wide Brain Activity From Local Recordings of Neural Potentials

Electrical recordings of neural activity from brain surface have been widely employed in basic neuroscience research and clinical practice for investigations of neural circuit functions, brain-computer interfaces, and treatments for neurological disorders. Traditionally, these surface potentials have been believed to mainly reflect local neural activity. It is not known how informative the locally recorded surface potentials are for the neural activities across multiple cortical regions.

To investigate that, simultaneous local electrical recording and wide-field calcium imaging in awake head-fixed mice can be performed. In some embodiments, a recurrent neural network model can be used to decode the calcium fluorescence activity of multiple cortical regions from local electrical recordings.

The mean activity of different cortical regions could be decoded from locally recorded surface potentials. Also, each frequency band of surface potentials differentially encodes activities from multiple cortical regions so that including all the frequency bands in the decoding model gives the highest decoding performance. Despite the close spacing between recording channels, surface potentials from different channels provide complementary information about the large-scale cortical activity and the decoding performance continues to improve as more channels are included. Finally, the decoding of whole dorsal cortex activity at pixel-level can be successfully performed using locally recorded surface potentials.

These results show that the locally recorded surface potentials indeed contain rich information of the large-scale neural activities, which could be further demixed to recover the neural activity across individual cortical regions. In some embodiments, the cross-modality inference approach may be adapted to virtually reconstruct cortex-wide brain activity, greatly expanding the spatial reach of surface electrical recordings without increasing invasiveness. Furthermore, it could be used to facilitate imaging neural activity across the whole cortex in freely moving animals, without requirement of head-fixed microscopy configurations.

As an important tool for electrophysiological recordings, neural electrodes implanted on the brain surface have been instrumental in basic neuroscience research to study large-scale neural dynamics in various cognitive processes, such as sensorimotor processing as well as learning and memory. In clinical settings, neural recordings have been adopted as a standard tool to monitor the brain activity in epilepsy patients before surgery for detection and localization of epileptogenic zones initiating seizures and functional cortical mapping. Neural activity recorded from the brain surface exhibits rich information content about the collective neural activities reflecting the cognitive states and brain functions, which is leveraged for various types of brain-computer interfaces during the past decade. For example, surface potential recordings have been used for studying motor control, such as controlling a screen cursor or a prosthetic hand. They have also been used to decode the mood of epilepsy patients, paving the way for the future treatment of neuropsychiatric disorders. Recent advances have shown that electrical recordings from cortical surface combined with the recurrent neural networks can even enable speech synthesis, demonstrating superior performance compared to those achieved through traditional noninvasive methods.

For the interpretation of surface potentials in terms of their neural correlates, most research has focused on local neural activities. The high-gamma band has been found to correlate with the ionic currents induced by synchronous synaptic input to the underlying neuron population. Besides that, the dendritic calcium spikes in the superficial cortical layers also contribute to surface potentials. Recently, it has been reported that even the action potentials of superficial cortical neurons could be detected in surface recordings. Despite the predominant focus of relating the surface potentials to local neural activity, they may also correlate with the large-scale activity of multiple cortical regions. This could be achieved through the intrinsic correlations of the spontaneous activities among large-scale cortical networks due to the anatomical connectivity and the global modulation of neuromodulatory projections. However, this rich information content of surface potentials encoded for the large-scale cortical activity remains unexploited and little is known about how local surface potentials are correlated with the spontaneous neural activities of distributed large-scale cortical networks.

In some embodiments of the disclosed technology, it can be determined whether the rich information content of the local neural potentials recorded from brain surface can be harnessed to infer the cortex-wide brain activity. The disclosed technology can be implemented based on some embodiments to employ optically transparent graphene microelectrodes implanted over the mouse somatosensory cortex and posterior parietal cortex (PPC) to perform simultaneous wide-field calcium imaging of the entire dorsal cortex during local neural recordings in awake mice. Multimodal datasets generated by these experiments are used to train a recurrent neural network model to learn the hidden spatiotemporal mapping between the local surface potentials and the cortex-wide brain activity detected by wide-field calcium imaging. In some embodiments, both the average spontaneous activity from multiple cortical regions and the pixel-level cortex-wide brain activity can be inferred from locally recorded surface potentials. The results show that in addition to the changes of local neural activity, the spontaneous fluctuations of locally recorded surface potentials also reflect the collective variations of large-scale neural activities across the entire cortex.

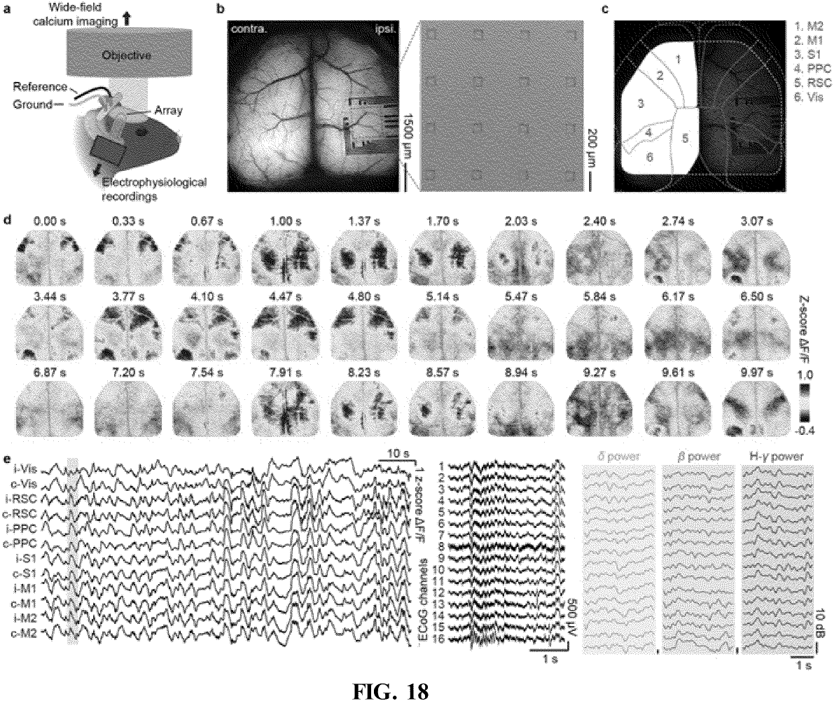

FIG. 18 shows simultaneous multimodal wide-field calcium imaging and surface potential recordings. FIG. 18(a) shows schematic of the multimodal experimental setup combining neural recordings using transparent graphene electrodes and wide-field calcium imaging. FIG. 18(b) shows example field of view of wide-field calcium imaging during experiment (left). Clear area at the center of the transparent array includes 16 graphene electrodes, whose scanning electron microscope image is shown on the right. FIG. 18(c) shows imaged cortical regions based on Allen Brain Atlas. M2: secondary motor cortex; M1: primary motor cortex; S1: primary somatosensory cortex; PPC: posterior parietal cortex; RSC: retrosplenial cortex; Vis: visual cortex. FIG. 18(d) shows wide-field fluorescence activity during 10 s long recordings, showing the diverse spontaneous activity across the mouse cortex. FIG. 18€ shows fluorescence activity for different cortical regions (left), the simultaneously recorded neural signals (middle) for a 3 s time interval (marked by the yellow bar on the left), and their power at three frequency bands (δ: 1-4 Hz, β: 15-30 Hz, Hy: 61-200 Hz, right three columns).

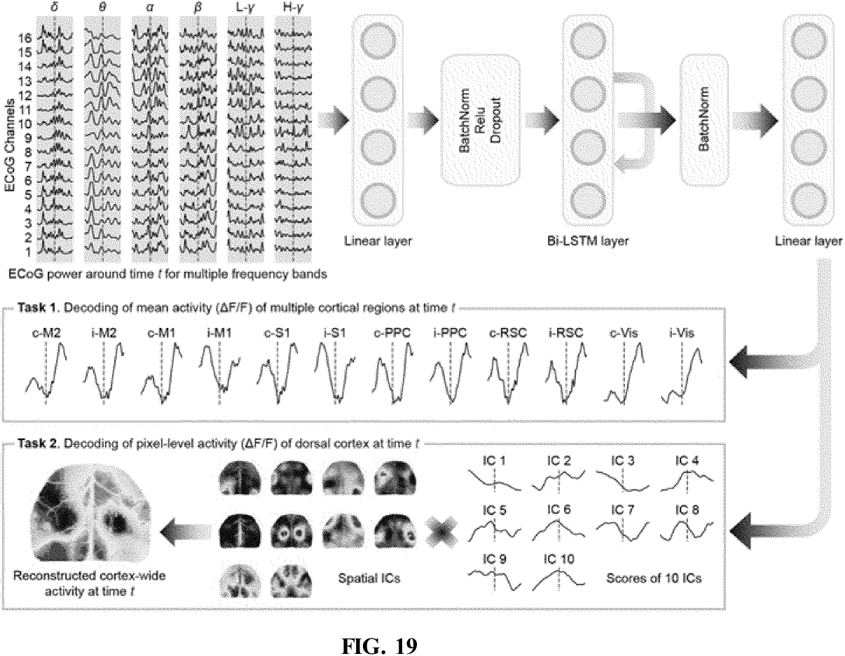

FIG. 19 shows schematic of the decoding model. Signal power from different channels during time interval [t - 1.5 s, t + 1.5 s] (90 time steps) is used to decode the cortical activity at time point t. The decoding neural network model consists of a sequential stacking of a linear hidden layer, one bidirectional LSTM (Bi-LSTM) layer and a linear readout layer. For the task of decoding the mean ΔF/F activity from multiple cortical regions, the final linear readout layer directly outputs the activities of 12 cortical regions at time t. For the task of decoding the pixel-level cortex-wide brain activity, the final linear readout layer outputs the weighting scores for all the Ics at time t, from which the cortex-wide brain activity at time t is reconstructed.

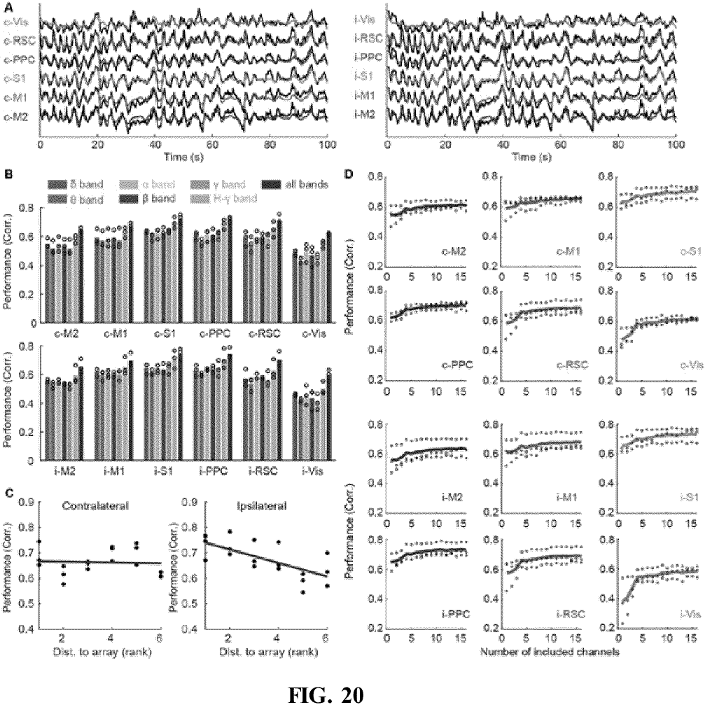

FIG. 20 shows decoding the activities of multiple cortical regions. FIG. 20(a) shows decoded (colorful) vs. ground truth (black) ΔF/F activity of different cortical regions in the contralateral (left) and ipsilateral (right) hemispheres for one mouse. FIG. 20(b) shows decoding performance evaluated for different cortical regions in the contralateral (top) and ipsilateral (bottom) hemispheres using different frequency bands (δ: 1-4 Hz, θ: 4-7 Hz, α: 8-15 Hz, β: 15-30 Hz, γ: 31-59 Hz, Hy: 61-200 Hz, and all six frequency bands). Each dot marks the mean correlation evaluated by ten-fold cross-validation using the data recorded from one mouse. FIG. 20(c) shows decoding performance for different cortical regions in the contralateral (left) and ipsilateral hemispheres evaluated as a function of distance (rank orders). Each dot is the mean correlation for one mouse given by ten-fold cross-validation. For ipsilateral hemisphere, the decoding performance decreases as the distance rank to the electrode array increases (ρ = -0.676, P = 0.002, n = 18). For contralateral hemisphere, no such correlation is observed (ρ = -0.163, P= 0.519, n = 18). Distances from the center of the array to the center of each cortical region: i-M2 3.63 mm, i-M1 2.65 mm, i-S1 0.98 mm, i-PPC 0.7 mm, i-RSC 2.36 mm, i-Vis 2.49 mm, c-M2 5.01 mm, c-M1 5.53 mm, c-S1 5.96 mm, c-PPC 5.37 mm, c-RSC 3.83 mm, c-Vis 6.32 mm. FIG. 20(d) shows decoding performance for different cortical regions in the contralateral (top) and ipsilateral (bottom) hemispheres using all the frequency bands, but different numbers of recording channels. Each dot marks the mean ten-fold cross-validated correlation over all the recording sessions for one mouse. Each line is the mean correlation averaged across three mice. For all the cortical regions, the decoding performance increases as more recording channels are included (P < 0.05, n =48, FDR correction).

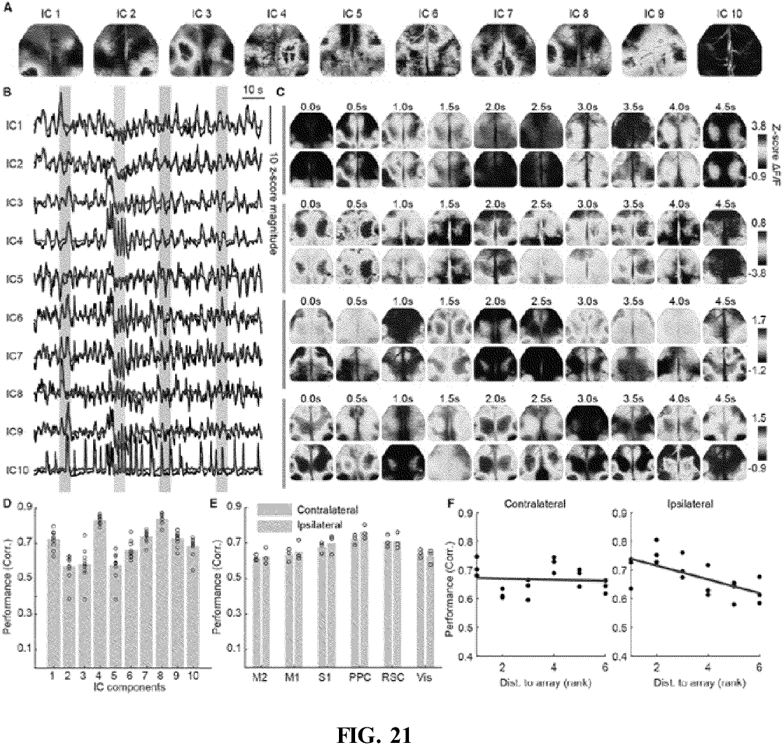

FIG. 21 shows decoding of the pixel-level cortex-wide brain activity. FIG. 21(a) shows identified Ics for the cortical activities recorded in one mouse, showing different functional modules of the cortical activity (IC 1-9) and the blood vessel activity (IC 10). FIG. 21(b) shows decoded (red) and ground truth (black) weighting scores of the observed cortex-wide activity onto the ten Ics shown in (a). FIG. 21(c) shows reconstructed (top rows) and ground truth (bottom rows) cortex-wide ΔF/F activity for four different time intervals, each lasting for 5 s, as indicated with different colors in FIG. 21(b). For visualization, the reconstructed and true cortex-wide brain activity are shown for every 0.5 s. FIG. 21(d) shows decoding performance evaluated for different Ics for one recording session. Each dot marks the decoding performance evaluated on one fold during the ten-fold cross-validation. The weighting scores for all the ten Ics could be successfully decoded. FIG. 21€ shows decoding performance evaluated at pixel-level for all the cortical regions in the ipsilateral and contralateral hemispheres. Each dot marks the mean ten-fold cross-validated correlation for individual pixels of one specific cortical region from one mouse. FIG. 21(f) shows pixel-wise decoding performance evaluated at individual cortical regions and displayed as a function of distance to the array (rank orders). For ipsilateral hemisphere, the decoding performance decreases as the distance to the electrode array increases (ρ = -0.649, P = 0.003, n = 18). For contralateral hemisphere, no correlation is observed (ρ = -0.074, P = 0.770, n = 18).

### Fabrication of Graphene Array

Electrode arrays are fabricated on 4″ silicon wafers spin coated with 20 µm thick polydimethylsiloxane (PDMS). 50 µm thick polyethylene terephthalate (PET) is placed on the adhesive PDMS layer and used as the array substrate. 10 nm of chromium and 100 nm of gold are deposited onto the PET using a sputtering system. The metal wires are patterned using photolithography and wet etching methods. Single-layer graphene is placed on the array area using a previously developed transfer process. The wafer is then soft baked for 5 min at 125° C. to better adhere graphene to the substrate. PMMA is removed via a 20 min acetone bath at room temperature then rinsed with isopropyl alcohol and DI water for ten 1 min cycles. The graphene channels are patterned using bilayer photolithography then oxygen plasma etched. A four-step cleaning method is performed on the array consisting of an AZ NMP soak, remover PG soak, acetone soak, and ten-cycle isopropyl alcohol/DI water rinse. 8 µm thick SU-8 2005 is spun onto the wafer as an encapsulation layer and openings are created at the active electrical regions using photolithography. The array is then given a final ten-cycle isopropyl alcohol/DI water rinse to clean SU-8 residue and baked for 20 min at temperature progressing from 125° C. to 135° C.

### Animals

All procedures are performed in accordance with protocols approved by the UCSD Institutional Animal Care and Use Committee and guidelines of the National Institute of Health. Mice (cross between CaMKIIa-tTA:B6;CBA-Tg(Camk2a-tTA)1Mmay/J [J AX 003010] and teto-GCaMP6s: B6;DBA-Tg(teto-GCaMP6s)2Niell/J [JAX 024742], Jackson laboratories) are group-housed in disposable plastic cages with standard bedding in a room with a reversed light cycle (12 h-12 h). Experiments are performed during the dark period. Both male and female healthy adult mice are used. Mice had no prior history of experimental procedures that could affect the results.

### Surgery and Multimodal Experiments

Adult mice (6 weeks or older) are anesthetized with 1% and 2% isoflurane and injected with baytril (10 mg kg⁻¹) and buprenorphine (0.1 mg kg⁻¹) subcutaneously. A circular piece of scalp is removed to expose the skull. After cleaning the underlying bone using a surgical blade, a custom-built head-bar is implanted onto the exposed skull over the cerebellum (~1 mm posterior to lambda) with cyanoacrylate glue and cemented with dental acrylic (Lang Dental). Two stainless-steel wires (791 900, A-M Systems) are implanted into the cerebellum as ground/reference. A craniotomy (~7 mm × 8 mm) is made to remove most of the dorsal skull and the graphene array is placed on the surface of one hemisphere, covering somatosensory cortex (S1) and PPC. The exposed cortex and the array are covered with a custom-made curved glass window, which is further secured with a tissue adhesive, cyanoacrylate glue and dental acrylic. Animals are fully awake before recordings. During recording, animals are head-fixed under the microscope, free to run or move their body, and not engaged in task.

The wide-field calcium imaging is performed using a commercial fluorescence microscope and a CMOS camera through the curved glass window as previously described. The light source for wide-field calcium imaging can be used. The filter set for imaging GCaMP signals is commercially installed in the microscope. It consists of a bandpass filter for the excitation light (485 ± 17 nm), a beamsplitter (500 nm), and a tunable bandpass filter centered at 520 nm for the emission light. Images are acquired using an imaging application at 29.98 Hz, 512 × 512 pixels (field of view: 8.5 mm × 8.5 mm, binning: 4, 16 bit).

The microelectrode array is connected to a custom-made connector board through a ZIF connector. The surface potential data is sampled with an amplifier and recorded using an amplifier system. The sampling frequency is 10 kHz. To synchronize the electrical recording with the calcium imaging, a trigger signal (TTL), a 2 V pulse of 1 s, can be used to trigger the start of the calcium imaging. Meanwhile, this trigger signal is also sent to the ADC of a recording system. During the data processing stage, the onset of the pulse can be detected and the imaging data and electrical data can be aligned to that time point. Three mice are recorded, each having two to three recording sessions. The length for each recording session is 1 h.

### ΔF/F Processing

To obtain the ΔF/F time series from the wide-field calcium imaging data, the 512 × 512 pixel images can be first down-sampled to smaller images of 128 × 128 pixels. For each pixel, a dynamic fluorescence (F) baseline for a given time point is defined as the 10th percentile value over 180 s around it. For the beginning and ending of each imaging block, the following and preceding 90 s window is used to determine the baseline, respectively. An 8th order 6 Hz Butterworth low-pass filter is applied to the ΔF/F activity of each pixel to remove the high frequency noise and hemodynamic contamination from heartbeat. The activity of each cortical region is obtained by averaging over the ΔF/F signals from all the pixels within the same cortical regions defined by the Allen Brain Atlas.

### Surface Recording Data Processing

The raw surface recording data is first passed through notch filters to eliminate the 60 Hz powerline contaminations and their higher harmonics at 120 Hz and 180 Hz. The signals are further filtered with multiple 6th order Butterworth band-pass filters designed for different frequency bands (δ: 1-4 Hz, θ: 4-7 Hz, α: 8-15 Hz, β: 15-30 Hz, γ: 31-59 Hz, Hy: 61-200 Hz). The resulting signals are squared and smoothed by a Gaussian function with 100 ms time window to obtain an estimate of the instantaneous power. To prepare the input data for the decoding neural network, the power traces at different frequency bands are down-sampled to 29.98 Hz by interpolation to match the sampling rate of calcium imaging data. To suppress the potential artifacts in the recording signal, at each frequency band we clip the power traces with a threshold of 95 percentile.

### Neural Network Models

The neural network model consists of a sequential stacking of a linear hidden layer, one bidirectional LSTM layer and a linear readout layer. The 1st linear layer is followed by batch normalization, ReLU activation, and dropout with a probability of 0.3. The LSTM layer is followed by batch normalization. The multi-channel power at different frequency bands are used as inputs to the network. To decode the neural activity at each time step t, the power segments between [t-1.5 s, t + 1.5 s] is used (90 time steps in total). The 1st linear layer had 16 neurons and the bidirectional LSTM had eight hidden neurons. The same neural network model is used for the two decoding tasks except that the number of neurons in the final output layer differs based on the targeting output. To decode the ΔF/F activity of 12 cortical regions simultaneously, the output neuron number is set to 12. To decode the cortex-wide brain activity, the output neuron number is set to ten to generate the scores for the ten ICs. Assuming using six frequency bands from 16 recording channels, setting sequence length of LSTM layer to 90, and setting batch size to 128, the input and output size for each layer of the model is shown in table 1. In some embodiments, the last two dimensions of the LSTM output can be flattened to make it 128 × 1440 before feeding it to the last linear layer.

TABLE 1

The size for input and output tensors of each layer

| | Input size | Output size |
|---|---|---|
| First linear layer | 128 × 90 × 96 | 128 × 90 × 16 |
| Bi-LSTM layer | 128 × 90 × 16 | 128 × 90 × 16 |
| Last linear layer | 128 × 1440 | 128 × 12 or 128 × 10 |

The neural network model is implemented in Pytorch. The model parameters are trained through Adam optimizer with learning rate = 1 × 10⁻⁴, beta1 = 0.9, beta2 = 0.999, epsilon = 1 × 10⁻⁸. The batch size is 128 and the training usually converged within ~30 epochs. For both tasks, the mean squared error is chosen as the loss function. The disclosed technology can be implemented in some embodiments to perform ten-fold cross-validation where each 1 h recording session is chunked into ten segments, each lasting for 6 min. The neural network model is trained on 9/10 of the data segments and tested on a different held-out segment that is unseen during the training. To evaluate the model performance, correlation between the decoded and ground truth data for each held-out set is averaged. For each 1 h recording session, a new network model is trained and tested. Then, for each mouse, the correlation is further averaged across the recording sessions to give the performance for that mouse.

### Statistical Tests

All statistical analyses are performed in MATLAB. Statistical tests are two-tailed and significance is defined by alpha pre-set to 0.05. All the statistical tests are described in the figure legends. Multiple comparisons are corrected for by Benjamini-Hochber corrections.

### Multimodal Recordings of Cortical Activity

Cortical recordings in both clinical applications and neuroscience studies use conventional metal-based neural electrode arrays. However, these opaque neural electrodes are not suitable for multimodal recordings combined with optical imaging since they will block the field of view and generate light-induced artifacts under optical imaging. Compared to conventional neural electrode arrays, graphene-based surface arrays are optically transparent and free from light-induced artifacts, both of which are key to the simultaneous electrical recordings and optical imaging of cortical activity. Wide-field calcium imaging is an optical imaging technique that can provide simultaneous monitoring of large-scale cortical activity and has been used to study the dynamics of multiple cortical regions and their coordination during behavior. Compared to fMRI that also offers large spatial coverage, the wide-field calcium imaging provides better spatiotemporal resolution and higher signal-to-noise ratio. It has been shown that wide-field calcium signals mainly reflect local neural activity. Therefore, the multimodal experiments combining electrical recordings based on graphene arrays and the wide-field calcium imaging generate unique datasets that are ideal for investigating the mapping from local neural signals to large-scale cortical activity.

The disclosed technology can be implemented in some embodiments to fabricate transparent graphene arrays on 50 µm thick flexible polyethylene terephthalate (PET) substrates (see section 2 for details). 10 nm of chromium and 100 nm of gold are deposited onto the PET and the metal wires are patterned using photolithography and wet etching methods. The graphene layer is transferred and patterned with photolithography and oxygen plasma etching to form electrode contacts. Finally, 8 µm thick SU-8 is used as an encapsulation layer and openings are created at the active electrical regions using photolithography. The graphene array has 16 recording channels, each of size 100 × 100 µm. The spacing between adjacent channels is 500 µm. The graphene array is implanted unilaterally over the somatosensory cortex (S1) and PPC of the mice to perform the simultaneous electrical recordings and wide-field calcium imaging (FIG. 18(a)). In some embodiments, multimodal recordings of spontaneous neural activity in awake mice can be performed during either quiet resting state or actively running or moving. An example wide-field image obtained during the experiment is shown in FIG. 18(b). Note that the cortical activity under the array could still be observed due to the transparency of the graphene electrode. Based on Allen brain atlas, the brain is parcellated into 12 different ipsilateral (the hemisphere with array implanted) and contralateral cortical regions (FIG. 18(c)), including the primary and secondary motor cortices (M1, M2), the somatosensory cortex (S1), the PPC, the retrosplenial cortex (RSC), and the visual cortex (Vis). Representative spontaneous cortical activity recorded during the experiment is shown in FIG. 18(d). In some embodiments, dynamical changes of large-scale cortical activity, involving co-activations of multiple cortical regions, can be observed. In the simultaneous multi-channel neural recordings, differences in power traces from different channels at multiple frequency bands during the spontaneous cortical activity (FIG. 18(e)) can also be observed. Compared with the fluorescence activity, the neural potential signal has a much higher temporal resolution and richer frequency components.

### Cortical Activity Decoder Design

To investigate whether the locally recorded surface potentials could be used to infer the cortex-wide brain activity, two decoding tasks, namely the decoding of the average activity from individual cortical regions and the decoding of pixel-level cortex-wide brain activity, can be investigated. To achieve these goals, the disclosed technology can be implemented in some embodiments to provide a compact neural network model consisting of a linear hidden layer, a one-layer LSTM network, and a linear readout layer (FIG. 19). In both tasks, the signal power traces of multiple frequency bands recorded from different recording channels are used as inputs to the neural network. In the 1st task, the neurons in the output layer of the neural network directly generate the activity of all the cortical regions simultaneously. In the 2nd task, the principal component analysis (PCA) can be performed on the cortical activity to remove the noise and reduce the dimensionality of the data. Across all the mice, the top ten PCs explain >92% variance in the data. Then based on the PCA results, spatial independent component analysis (ICA) can be further performed to obtain the independent components (ICs) and their weighting scores for the data at each time frame. In all the three mice, the identified ICs reflect different functional modules and hemodynamic signals on blood vessels and provide a set of functionally meaningful basis for the decomposition of the large-scale cortical activity. The output layer of the neural network directly generates the estimated weighting scores of individual ICs, which are further used to reconstruct the cortex-wide brain activity at each time frame with pixel-level spatial resolution (FIG. 19).

### Decoding of Activity for Individual Cortical Regions

Based on the multimodal data collected during the animal experiment and the above designed decoder network model, the mean activity of both the ipsilateral and contralateral cortical regions can be decoded using the power of six frequency bands from all recording channels. An example of decoded and ground truth (ΔF/F from wide-field calcium imaging) cortical activity from one held-out set is shown in FIG. 20(a). The decoding performances for S1, PPC, and RSC regions closely resemble the ground truth cortical activity, while the decoding performances for M1, M2, and Vis are lower, possibly due to their increasing distances to the recording electrode array. In some embodiments, the same decoding analysis can be performed using shuffled data. The results show decoding performance close to zero. In some embodiments, the stability of the decoding performance across time using a 30 s sliding window can be evaluated. The results show that the decoding performance fluctuates from time to time but remains stable in the longer time intervals. In some embodiments, decoding performance of individual cortical regions during rest and movement intervals and found similar decoding performance between rest and movement phases can be compared. Therefore, the fluctuations of the decoding performance across time are not due to animal movements.

To further evaluate how informative different frequency bands are for the decoding of the activity from different cortical regions, the signal power from different frequency bands of all channels can be used as inputs and ten-fold cross-validation can be performed to evaluate the decoding performance of the neural network model. In some embodiments, even though all the frequency bands are informative of the activities in different cortical regions, the high gamma power band gives the highest decoding performance for all the cortical regions compared to other frequency bands. However, across all the cortical regions, using all the frequency bands yields the best decoding performance compared to using any single frequency band (FIG. 20(b)), implying that different frequency bands provide complementary information about the activity in multiple cortical regions. Decoding with the shuffled data gives performance close to zero for all the frequency bands. For the ipsilateral cortical regions, there can be a negative correlation between their decoding performance and their distance ranks to the recording array. However, for the contralateral cortical regions, no significant correlation is observed (FIG. 20(c)). When comparing the decoding results of the activity from ipsilateral cortical regions using different frequency bands, higher frequency bands tend to have a steeper slope for the decoding performance vs. distance to the recording array.

Besides the frequency bands, it can be determined whether different recording channels encode nonredundant information for decoding the activity of different cortical regions. Therefore, the decoding performance of the neural network model can be evaluated using all six frequency bands from different numbers of channels. Specifically, ten-fold cross-validation can be performed on the neural network multiple times and each time the signal power of all frequency bands can be sequentially added from one random channel until all the channels are included. As shown in FIG. 20(d), for all the cortical regions, increasing the number of channels significantly improves the decoding performance, suggesting that recording channels of local neural potentials provide nonredundant information about the activity from multiple cortical regions. On the other hand, decoding with the shuffled data gives performance close to zero for different number of included channels.

### Decoding of Pixel-Wise Activity Across Cortex

Given that the local neural signals encode average activity from individual cortical regions, which could be recovered by the neural network model using multi-channel signal power of different frequency bands, it can be determined whether the pixel-level activity across the whole dorsal cortex could also be decoded using locally recorded neural signals. The same neural network model for decoding the average activity in different cortical regions is then employed to simultaneously decode the ten IC scores at each time frame. The power traces of all the six frequency bands from all the recording channels are used as inputs to the neural network. An example of the decoded and ground truth scores for the ten ICs from one held-out set is shown in FIG. 21(B). The decoding result can be obtained using shuffled data. Based on the decoded IC scores and the IC modules (FIG. 21(a)), the pixel-level cortex-wide activity at each time frame could be reconstructed. Examples of the reconstructed pixel-level cortex-wide activity during four representative time intervals are shown in FIG. 21(c). The reconstructed cortex-wide activity captured various patterns of cortical activations in ground truth, including both the synchronous and asynchronous activations among different cortical regions. These diverse activation patterns cannot be explained solely by PC1 (see FIG. 21(c)). To further quantify this observation, the correlation between the ground truth activity of each ICs and the PC1 can be computed. The median correlations between IC1, IC2 and IC8 to PC1 are close to zero, showing that their activities are not strongly correlated to PC1. These results suggest that the model does not merely predict dominant activity patterns showing activation around S1 and RSC. In some embodiments, all the ten IC scores could be decoded using the locally recorded neural signals (FIG. 21(d)). In some embodiments, the pixel-level cortex-wide activity could be reconstructed for all the recording sessions. This reveals that the cortical activations of distinct functional modules indeed induce different responses in local cortical electrical signals, which could be in turn used to recover the diverse cortex-wide activity patterns. In addition to cortical activity, in all the mice, one or two ICs can show the hemodynamic activity. The decoding results also show that these hemodynamic activities could be decoded from the neural recordings, which is mainly due to the fact that hemodynamic activity and the neural activity are often correlated. Next, the pixel-level correlations between the decoded and ground truth activities imaged using wide-field imaging in individual cortical regions can be examined. In some embodiments, there can be high correlations between the decoded and the ground truth data for all cortical regions (FIG. 21(e)) and close-to-zero correlations using shuffled data. The activities of cortical regions closer to the array are better decoded than those of the cortical regions far away from the array. Consistent with the decoding of mean activity in each cortical region, the pixel-wise correlation decreases as the distance rank to the surface array increases for the ipsilateral cortical regions, whereas for the contralateral cortical regions no such correlation exists (FIG. 21(f)).

In some embodiments, multimodal recordings of local neural potentials and wide-field calcium imaging in awake mice can be performed and a recurrent neural network model can be developed to decode the large-scale spontaneous cortical activity from the locally recorded multi-channel electrical signals. Both the averaged and the pixel-level activity across the entire dorsal cortex could be decoded, and the best decoding performance is achieved using all frequency bands of recorded neural potentials. These results suggest that even though the cortical electrical recording is a complex signal contributed by various mechanisms at multiple spatial scales, the responses in individual frequency bands across multiple recording channels still provide important discriminative information about the activity of different cortical regions. By developing a decoder model, the mixed information in the electrical signal responses could be used to recover the simultaneously recorded cortex-wide brain activity.

The cortical potentials have long been believed to mainly detect local neural activities that are within a sensing distance between 500 µm to 1-3 mm, depending on the size of the electrode as well as the spatial correlation pattern of neural activity. Consistent with this claim, for the decoding of mean activity from individual cortical regions, there can be a decreasing decoding performance for the ipsilateral cortical regions located ~1.5-3 mm from the array. Interestingly, for the contralateral cortical regions, the decoding is still possible even though their activities are unlikely to be directly detected by the neural electrodes. In some embodiments, the successful decoding of contralateral cortical regions is mainly due to the fact that the spontaneous activities of same functional cortical regions in both hemispheres are often correlated. Such correlated activity could arise from the anatomical connectivity and further orchestrated by neuromodulatory projections.

In some embodiments, the decoding results for the activity of individual cortical regions show that even with single recording channel, the decoding is possible (mean correlation performance between 0.35 and 0.65 for different cortical regions). By including more channels, initially an increase is observed in decoding performance, but the performance starts to saturate after the inclusion of ten recording channels (mean correlation performance between 0.6 and 0.75 for different cortical regions). In some embodiments, this is mainly because of the fact that the neural potentials in adjacent channels are partially correlated due to the volume conduction in the brain tissue. It has been shown that the correlation between neural potentials from adjacent channels at different frequency bands decreases as the distance increases. Even though the cross-channel correlation at high frequency bands is lower than that at low frequency bands, it does not go below chance level even with a distance of ~1.5 mm. However, the results empirically confirm that even though the neural potentials from adjacent channels are partially correlated, they still differentially encode information about the cortical activities to some extent so that sequentially including more recording channels tends to increase the decoding performance. However, beyond a certain threshold adding more channels does not further increase the decoding performance.

For the decoding of cortex-wide brain activity, instead of attempting to directly reconstruct the activity of individual pixels, the disclosed technology can be implemented in some embodiments to perform PCA followed by spatial ICA on the cortical activity and later to decode IC scores to recover the cortex-wide activity at pixel level. The adoption of this approach is based on both scientific and computational considerations. First, the PCA effectively reduced the spatial dimensions, while preserving a large proportion of variance in cortical activity. Since the activity of each single pixel is noisy, performing PCA reduced the noise, leading to a more reliable estimate of the true activity. Second, choosing the IC scores as network outputs greatly reduced the parameters in the output layer of the neural network model, prevented overfitting, and speeded up model training. Finally, the spontaneous cortex-wide brain activity is decomposed into a set of local and spatially organized cortical activation patterns based on neural activity, generating a biologically meaningful decomposition that matches the brain dynamics. This decomposition provides a good demixing of cortex-wide brain activity and enables a tractable mapping from cortical neural responses, which can be learned by the decoding network model. Taken together, these results reveal that the activation of different cortical functional modules are associated with distinct components in local neural activity. By exploiting the mapping between the two modalities, the decoding of cortex-wide brain activity is possible from locally recorded neural signals.

The disclosed technology can be implemented in some embodiments to provide a neural network model that can show that both the mean activity of different cortical regions and the pixel-level cortex-wide neural activity can be decoded using locally recorded surface potentials. These findings demonstrated that the locally recorded neural potentials indeed contain rich information for large-scale neural activity and the surface potential responses in different frequency bands and different recording channels provide distinct information about the large-scale neural activity.

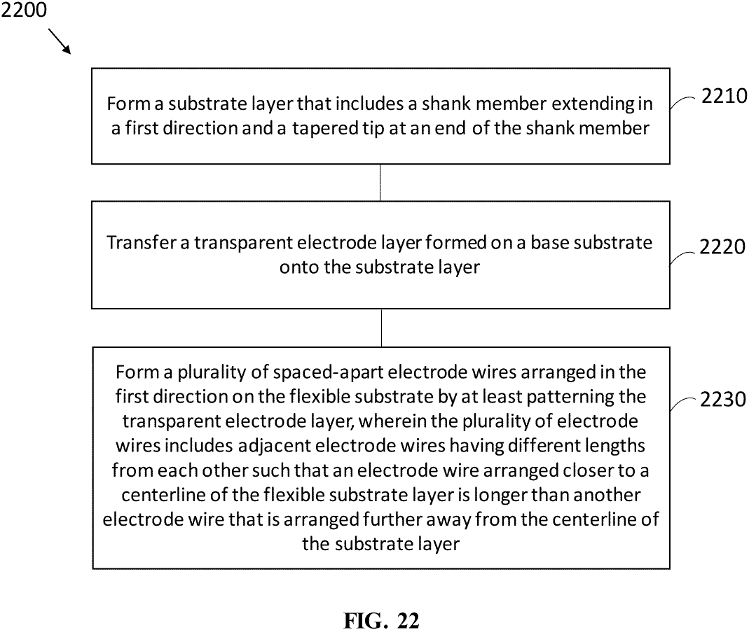

FIG. 22 shows an example method of fabricating a microelectrode array based on some embodiments of the disclosed technology.

In some implementations, a method 2200 includes, at 2210, forming a substrate layer that includes a shank member extending in a first direction and a tapered tip at an end of the shank member, at 2220, transferring a transparent electrode layer formed on a base substrate onto the substrate layer, and at 2230, forming a plurality of spaced-apart electrode wires arranged in the first direction on the flexible substrate by at least patterning the transparent electrode layer, wherein the plurality of electrode wires includes adjacent electrode wires having different lengths from each other such that an electrode wire arranged closer to a centerline of the flexible substrate layer is longer than another electrode wire that is arranged further away from the centerline of the substrate layer.

In some implementations, a substrate layer may include a plurality of shank members, each of which includes a tapered tip at an end of each shank member. In one example, the shank members extend in the same direction. In another example, some of the shank members extend in a direction different from other shank members. In some implementations, a substrate layer may include 4-8 shank members. In one example, each shank can have 64-128 electrodes, and thus the total electrode number may be 512-1024.

Therefore, various implementations of features of the disclosed technology can be made based on the above disclosure, including the examples listed below.

Example 1. A microelectrode array comprising: a flexible substrate layer including a shank member extending in a first direction and a tapered tip at an end of the shank member; and a plurality of electrode wires arranged in the first direction on the flexible substrate layer, wherein the plurality of electrode wires includes adjacent electrode wires having different lengths from each other such that an electrode wire arranged closer to a centerline of the flexible substrate layer is longer than an adjacent electrode arranged further away from the centerline of the flexible substrate.

In some implementations, a flexible substrate layer may include a plurality of shank members. Each of the shank members includes a tapered tip at an end thereof. In one example, the plurality of shank members extends in one direction. In another example, some of the shank members extend in a direction different from other shank members.

Example 2. The microelectrode array of example 1, further comprising an encapsulation layer disposed over the plurality of electrode wires.

Example 3. The microelectrode array of example 2, wherein the encapsulation layer includes one or more electrode openings structured to expose a portion of one or more electrode wires.

Example 4. The microelectrode array of example 3, wherein the one or more electrode openings are arranged along the tapered tip.

Example 5. The microelectrode array of example 3, wherein the one or more electrode openings are spaced apart from the tapered tip.

Example 6. The microelectrode array of example 5, wherein the electrode opening corresponding to the electrode wire arranged closer to the centerline of the flexible substrate layer is spaced apart from the tapered tip by a first distance, and the electrode opening corresponding to another electrode wire is spaced apart from the tapered tip by a second distance, wherein the first distance is shorter than the second distance.

Example 7. The microelectrode array of example 1, wherein the plurality of electrode wires is arranged in the first direction at a uniform interval.

Example 8. The microelectrode array of example 1, wherein the flexible substrate layer includes a transparent material.

Example 9. The microelectrode array of example 1, wherein the plurality of electrode wires includes optically transparent graphene microelectrodes.

Example 10. The microelectrode array of example 1, wherein the flexible substrate layer includes a polyethylene terephthalate (PET) substrate.

Example 11. A method of fabricating a microelectrode array, comprising: forming a substrate layer that includes a shank member extending in a first direction and a tapered tip at an end of the shank member; transferring a transparent electrode layer formed on a base substrate onto the substrate layer; and forming a plurality of spaced-apart electrode wires arranged in the first direction on the substrate layer by at least patterning the transparent electrode layer, wherein the plurality of electrode wires includes adjacent electrode wires having different lengths from each other such that an electrode wire arranged closer to a centerline of the substrate layer is longer than another electrode wire that is arranged further away from the centerline of the substrate layer.

In some implementations, a substrate layer may include a plurality of shank members, each of which includes a tapered tip at an end of each shank member. In one example, the shank members extend in the same direction. In another example, some of the shank members extend in a direction different from other shank members.

Example 12. The method of example 11, further comprising: forming an encapsulation layer over the plurality of electrode wires; and forming one or more electrode openings on the encapsulation layer to expose a portion of one or more electrode wires.

Example 13. The method of example 11, wherein forming the substrate layer includes: forming a polydimethylsiloxane (PDMS) layer on a silicon substrate; and forming a polyethylene terephthalate (PET) layer on the PDMS layer.

Example 14. The microelectrode array of example 11, wherein transferring the transparent electrode layer onto the substrate layer includes transferring a graphene layer onto the substrate layer.

Example 15. A microelectrode array comprising: a flexible substrate layer extending in a first direction and including a tapered tip at an end of the flexible substrate layer; a plurality of electrode wires arranged in the first direction at an interval on the flexible sub strate layer, wherein the plurality of electrode wires includes a first electrode wire arranged along a centerline of the flexible substrate layer and a second electrode wire arranged along an edge of the flexible substrate layer, wherein the first electrode wire is longer than the second electrode wire; and an encapsulation layer disposed over the plurality of electrode wires and including one or more electrode openings structured to expose a portion of one or more electrode wires.

Example 16. The microelectrode array of example 15, wherein the one or more electrode openings are arranged along the tapered tip.

Example 17. The microelectrode array of example 16, wherein the electrode opening of the first electrode wire is spaced apart from the tapered tip by a first distance, and the electrode opening of the second electrode wire is spaced apart from the tapered tip by a second distance, wherein the first distance is shorter than the second distance.

Example 18. The microelectrode array of example 15, wherein the flexible substrate layer includes a transparent material.

Example 19. The microelectrode array of example 15, wherein the plurality of electrode wires includes optically transparent graphene microelectrodes.

Example 20. The microelectrode array of example 15, wherein the flexible substrate layer includes a flexible polyethylene terephthalate (PET) substrate.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A microelectrode array comprising:
a flexible substrate layer including a shank member extending in a first direction and a tapered tip at an end of the shank member; and
a plurality of electrode wires arranged in the first direction on the flexible substrate layer, wherein the plurality of electrode wires includes adjacent electrode wires having different lengths from each other such that an electrode wire arranged closer to a centerline of the flexible substrate layer is longer than an adjacent electrode arranged further away from the centerline of the flexible substrate.

2. The microelectrode array of claim 1, further comprising an encapsulation layer disposed over the plurality of electrode wires.

3. The microelectrode array of claim 2, wherein the encapsulation layer includes one or more electrode openings structured to expose a portion of one or more electrode wires.

4. The microelectrode array of claim 3, wherein the one or more electrode openings are arranged along the tapered tip.

5. The microelectrode array of claim 3, wherein the one or more electrode openings are spaced apart from the tapered tip.

6. The microelectrode array of claim 5, wherein the electrode opening corresponding to the electrode wire arranged closer to the centerline of the flexible substrate layer is spaced apart from the tapered tip by a first distance, and the electrode opening corresponding to another electrode wire is spaced apart from the tapered tip by a second distance, wherein the first distance is shorter than the second distance.

7. The microelectrode array of claim 1, wherein the plurality of electrode wires is arranged in the first direction at a uniform interval.

8. The microelectrode array of claim 1, wherein the flexible substrate layer includes a transparent material.

9. The microelectrode array of claim 1, wherein the plurality of electrode wires includes optically transparent graphene microelectrodes.

10. The microelectrode array of claim 1, wherein the flexible substrate layer includes a polyethylene terephthalate (PET) substrate.

11. A method of fabricating a microelectrode array, comprising:
forming a substrate layer that includes a shank member extending in a first direction and a tapered tip at an end of the shank member;
transferring a transparent electrode layer formed on a base substrate onto the substrate layer; and
forming a plurality of spaced-apart electrode wires arranged in the first direction on the substrate layer by at least patterning the transparent electrode layer, wherein the plurality of electrode wires includes adj acent electrode wires having different lengths from each other such that an electrode wire arranged closer to a centerline of the substrate layer is longer than another electrode wire that is arranged further away from the centerline of the substrate layer.

12. The method of claim 11, further comprising:
forming an encapsulation layer over the plurality of electrode wires; and
forming one or more electrode openings on the encapsulation layer to expose a portion of one or more electrode wires.

13. The method of claim 11, wherein forming the substrate layer includes:
forming a polydimethylsiloxane (PDMS) layer on a silicon substrate; and
forming a polyethylene terephthalate (PET) layer on the PDMS layer.

14. The microelectrode array of claim 11, wherein transferring the transparent electrode layer onto the sub strate layer includes transferring a graphene layer onto the sub strate layer.

15. A microelectrode array comprising:
a flexible substrate layer extending in a first direction and including a tapered tip at an end of the flexible substrate layer;
a plurality of electrode wires arranged in the first direction at an interval on the flexible substrate layer, wherein the plurality of electrode wires includes a first electrode wire arranged along a centerline of the flexible substrate layer and a second electrode wire arranged along an edge of the flexible substrate layer, wherein the first electrode wire is longer than the second electrode wire; and
an encapsulation layer disposed over the plurality of electrode wires and including one or more electrode openings structured to expose a portion of one or more electrode wires.

16. The microelectrode array of claim 15, wherein the one or more electrode openings are arranged along the tapered tip.

17. The microelectrode array of claim 16, wherein the electrode opening of the first electrode wire is spaced apart from the tapered tip by a first distance, and the electrode opening of the second electrode wire is spaced apart from the tapered tip by a second distance, wherein the first distance is shorter than the second distance.

18. The microelectrode array of claim 15, wherein the flexible substrate layer includes a transparent material.

19. The microelectrode array of claim 15, wherein the plurality of electrode wires includes optically transparent graphene microelectrodes.

20. The microelectrode array of claim 15, wherein the flexible substrate layer includes a flexible polyethylene terephthalate (PET) substrate.
